(19) United States
(12) Patent Application Publication
(10) Pub. No.: US 2024/0173344 A1
(43) Pub. Date: May 30, 2024

(54) USE OF GABAA RECEPTOR AS TARGET IN PREPARATION OR SCREENING OF DRUG FOR LOWERING BLOOD LIPID LEVEL, TREATING OBESITY, AND/OR IMPROVING METABOLISM

(71) Applicants: SHANGHAI INSTITUTE OF ENDOCRINE AND METABOLIC DISEASES, Shanghai (CN); RUIJIN HOSPITAL, SHANGHAI JIAO TONG UNIVERSITY SCHOOL OF MEDICINE, Shanghai (CN)

(72) Inventors: Guang NING, Shanghai (CN); Jiqiu WANG, Shanghai (CN); Qianqian LYU, Shanghai (CN); Wenzhi XUE, Shanghai (CN); Ruixin LIU, Shanghai (CN)

(73) Assignees: SHANGHAI INSTITUTE OF ENDOCRINE AND METABOLIC DISEASES, Shanghai (CN); RUIJIN HOSPITAL, SHANGHAI JIAO TONG UNIVERSITY SCHOOL OF MEDICINE, Shanghai (CN)

(21) Appl. No.: 18/127,421

(22) Filed: Mar. 28, 2023

(30) Foreign Application Priority Data
Nov. 22, 2022 (CN) 2022114640370

(51) Int. Cl. A61K 31/7048; A61K 31/5517; A61P 3/04

(52) U.S. Cl. CPC A61K 31/7048; A61K 31/5517; A61P 3/04

(57) ABSTRACT

The present disclosure belongs to the technical field of medicine, and relates to a use of a GABA_{A} receptor as a target in the preparation or screening of a drug for reducing blood lipid, treating obesity, and/or improving metabolism. The present disclosure provides a use of a GABA_{A} receptor as a target in the preparation or screening of a drug with one or more functions selected from the group consisting of functions (1) to (4): (1) reducing blood lipid; (2) treating obesity; (3) improving metabolic syndrome; and (4) inhibiting small intestinal lipid absorption. The GABA_{A} receptor is closely related to a lipid absorption ability of a small intestine, and the use of the present disclosure provides a new drug target and a new therapeutic means for treating obesity, reducing blood lipid, and improving metabolism.

### CROSS-REFERENCE TO RELATED APPLICATION

This patent application claims the benefit and priority of Chinese Patent Application No. 202211464037.0 filed with the China National Intellectual Property Administration on Nov. 22, 2022, the disclosure of which is incorporated by reference herein in its entirety as part of the present application.

### TECHNICAL FIELD

The present disclosure belongs to the technical field of medicine, and specifically relates to use of a GABA_{A} receptor as a target in the preparation or screening of a drug for reducing blood lipid, treating obesity, and/or improving metabolism.

### BACKGROUND

Obesity is a risk factor causing various diseases such as diabetes, cardiovascular disease (CVD), and cancer, and seriously jeopardizes the human health. With the improvement of people's living conditions and the change of people's dietary structures, the global obese population is growing rapidly. However, there are relatively limited means for treating obesity and effectively losing weight at present. Therefore, in order to improve people's life quality and health status and prevent chronic diseases, it is urgent to explore new strategies for treating obesity and develop safe and effective obesity interventions 1-4.

### CITED REFERENCES

1 Pan, X. F., Wang, L. & Pan, A. Epidemiology and determinants of obesity in China. Lancet Diabetes Endocrinol 9, 373-392, doi:10.1016/S2213-8587(21)00045-0 (2021).
2 Zeng, Q., Li, N., Pan, X. F., Chen, L. & Pan, A. Clinical management and treatment of obesity in China. Lancet Diabetes Endocrinol 9, 393-405, doi:10.1016/S2213-8587(21)00047-4 (2021).
3 Collaboration, N. C. D. R. F. Trends in adult body-mass index in 200 countries from 1975 to 2014: a pooled analysis of 1698 population-based measurement studies with 19.2 million participants. Lancet 387, 1377-1396, doi:10.1016/S0140-6736(16)30054-X (2016).
4 Lu, J., Bi, Y. & Ning, G. Curbing the obesity epidemic in China. Lancet Diabetes Endocrinol 4, 470-471, doi:10.1016/S2213-8587(16)30007-9 (2016).

### SUMMARY

The present disclosure is intended to provide a use of a GABA_{A} receptor as a target in the preparation or screening of a drug for reducing blood lipid, treating obesity, and/or improving metabolism. The GABA_{A} receptor is closely related to a lipid absorption ability of a small intestine, and the use according to the present disclosure provides a new drug target and a new therapeutic means for treating obesity, reducing blood lipid, and improving metabolism.

The present disclosure provides a use of a GABA_{A} receptor as a target in the preparation or screening of a drug with any one or more functions selected from the group consisting of functions (1) to (4): (1) reducing blood lipid; (2) treating obesity; (3) improving metabolic syndrome; and (4) inhibiting small intestinal lipid absorption.

The present disclosure also provides a use of a substance for increasing an expression level of a GABA_{A} receptor in the preparation of a drug with one or more functions selected from the group consisting of functions (1) to (4): (1) reducing blood lipid; (2) treating obesity; (3) improving metabolic syndrome; and (4) inhibiting small intestinal lipid absorption.

The present disclosure also provides a use of a substance for improving sensitivity of a GABA_{A} receptor or a chloride channel opening frequency, increasing a chloride influx, or causing nerve cell hyperpolarization in the preparation of a drug with one or more functions selected from the group consisting of functions (1) to (4): (1) reducing blood lipid; (2) treating obesity; (3) improving metabolic syndrome; and (4) inhibiting small intestinal lipid absorption.

The present disclosure also provides a use of a GABA_{A} receptor agonist and/or allosteric modulator in the preparation of a drug with one or more functions selected from the group consisting of functions (1) to (4): (1) reducing blood lipid; (2) treating obesity; (3) improving metabolic syndrome; and (4) inhibiting small intestinal lipid absorption.

In some embodiments, the GABA_{A} receptor agonist includes puerarin and/or a derivative thereof, or a benzodiazepine; and the benzodiazepine includes one or more selected from the group consisting of flunitrazepam, diazepam, triazolam, and flumazenil.

The present disclosure also provides a use of a substance for inhibiting nerve excitability of the dorsal motor nucleus of the vagus (DMV) in the preparation of a drug with one or more functions selected from the group consisting of functions (1) to (4): (1) reducing blood lipid; (2) treating obesity; (3) improving metabolic syndrome; and (4) inhibiting small intestinal lipid absorption.

In some embodiments, the substance for inhibiting nerve excitability of the DMV includes a substance for increasing an expression level of a GABA_{A} receptor or a substance for improving sensitivity of a GABA_{A} receptor or a chloride channel opening frequency, increasing a chloride influx, or causing nerve cell hyperpolarization.

The present disclosure also provides a use of puerarin and/or a derivative thereof in the preparation of a drug with one or more functions selected from the group consisting of functions (1) to (4): (1) reducing blood lipid; (2) treating obesity; (3) improving metabolic syndrome; and (4) inhibiting small intestinal lipid absorption.

The present disclosure also provides a use of a gene Gabra1 and/or a gene Gabrg2 as a drug target in the screening of a drug with one or more functions selected from the group consisting of functions (1) to (4): (1) reducing blood lipid; (2) treating obesity; (3) improving metabolic syndrome; and (4) inhibiting small intestinal lipid absorption.

The present disclosure provides a use of a GABA_{A} receptor as a target in the preparation or screening of a drug for reducing blood lipid, treating obesity, and/or improving metabolism. At present, there is no research report internationally that the GABA_{A} receptor in DMV has the effects of inhibiting small intestinal lipid absorption, reducing blood lipid, treating obesity, and improving metabolism. The present disclosure proposes for the first time that the GABA_{A} receptor may serve as a drug target for treating obesity, reducing blood lipid, inhibiting small intestinal lipid absorption, and improving metabolism. The present disclosure provides a use of a GABA_{A} receptor (a receptor including α1/γ2 subunits) in DMV as a drug target for lipid-lowering, weight-losing, and metabolism-improving. Investigation results of the present disclosure show that puerarin (and a derivative thereof) is an agonist and allosteric modulator for the GABA_{A} receptor; and puerarin (and a derivative thereof) may inhibit the nerve excitability of DMV by activating the brainstem GABA_{A} receptor, such that the lipid absorption ability of the small intestine is inhibited to reduce blood lipid and body weight (treating obesity), and improve metabolism, which provides experimental data and theoretical basis for the research and development of the weight-reducing aid.

### BRIEF DESCRIPTION OF THE DRAWINGS

To describe the technical solutions in the examples of the present disclosure or in the prior art more clearly, the accompanying drawings required in the examples are briefly introduced below. Apparently, the accompanying drawings in the following description show merely some examples of the present disclosure, and a person of ordinary skill in the art may still derive other drawings from these accompanying drawings without creative efforts.

FIGS. 1A-1E show a regulation effect of the DMV on the small intestinal lipid absorption that is demonstrated by the chemogenetic strategy of the present disclosure, where FIG. 1A is an experimental flow chart of the chemogenetic strategy to inhibit neurons in the DMV; FIG. 1B shows body weight curves of the experimental group and the control group after the inhibition of the chemogenetic strategy on neurons in the DMV; FIG. 1C shows blood lipid level changes after the DMV is inhibited; FIG. 1D shows excreted fecal TG results before and after the intervention by the chemogenetic strategy; and FIG. 1E shows the inhibition of the chemogenetic strategy on the absorption of lipid nutrients such as TG in the jejunum.

FIGS. 2A-2B show an inhibitory effect of puerarin on neurons in the DMV obtained through electrophysiological recording of brain slices provided in the present disclosure, where FIG. 2A is a demonstration of an electrophysiological operation of a brain slice (there is a green-fluorescent dye in a pipette solution of a recording electrode to label recorded neuronal cells); and FIG. 2B shows electrophysiological results of brain slices (action potentials of neurons in the DMV during puerarin incubation and after elution are recorded).

FIGS. 3A-3F shows the effects of puerarin to inhibit the small intestinal lipid absorption and reduce the body weight in the obese mouse model provided by the present disclosure, where FIG. 3A is a schematic diagram illustrating a process of intraperitoneal injection of puerarin into the obese mouse model; FIG. 3B shows the c-fos immunofluorescence staining results of brain slices, where a fluorescence signal represents the neuronal activity; FIG. 3C shows body weight curves of obese mouse models in the puerarin experimental group and the control group; FIG. 3D shows the quantification results of blood lipid levels of the puerarin experimental group and the control group; E shows the measurement results of jejunal TG levels of the puerarin experimental group and the control group; and F shows the representative oil red O staining results, which visually present the absorption of lipid nutrients in a small intestine.

FIG. 4 shows the colocalization staining results of the puerarin provided by the present disclosure with the GABA_{A} receptor in the DMV.

FIGS. 5A-5B show the cryo-EM analysis results of a drug binding site of the puerarin provided by the present disclosure to the GABA_{A} receptor, where FIG. 5A is a cryo-EM structural diagram illustrating the binding of puerarin to the GABA_{A} (α1β3γ2) receptor, and FIG. 5B shows the electrical signal changes.

FIGS. 6A-6F show that the specific knockdown of Gabra1 provided by the present disclosure can block an inhibitory effect of puerarin on neurons in the DMV and the effects of puerarin to reduce the body weight, lower the blood lipid level, and improve the metabolism, where FIG. 6A is a schematic diagram illustrating a process of intraperitoneal injection of puerarin into an obese mouse model in which Gabra1 undergoes DMV-specific knockdown; FIG. 6B shows the c-fos immunofluorescence staining results of brain slices; FIG. 6C shows the quantitative statistical results of c-fos staining signals in the DMV; FIG. 6D shows body weight curves of obese mouse models in the control group, the puerarin group, and the Gα blocking group; FIG. 6E shows the quantitative measurement results of blood lipid levels in the control group, the puerarin group, and the Gα blocking group; and FIG. 6F shows the measurement results of jejunal TG levels in the control group, the puerarin group, and the Gα blocking group.

FIGS. 7A-7F shows that the specific knockdown of Gabrg2 provided by the present disclosure can block an inhibitory effect of puerarin on neurons in the DMV and the effects of puerarin to reduce the body weight, lower the blood lipid level, and improve the metabolism, where FIG. 7A is a schematic diagram illustrating a process of intraperitoneal injection of puerarin into an obese mouse model in which Gabrg2 undergoes DMV-specific knockdown; FIG. 7B shows the c-fos immunofluorescence staining results of brain slices; FIG. 7C shows the quantitative statistical results of c-fos staining signals in the DMV; FIG. 7D shows body weight curves of obese mouse models in the control group, the puerarin group, and the Gγ2 blocking group; FIG. 7E shows the quantitative measurement results of blood lipid levels in the control group, the puerarin group, and the Gγ2 blocking group; and FIG. 7F shows the measurement results of jejunal TG levels in the control group, the puerarin group, and the Gγ2 blocking group.

FIGS. 8A-8L show the effects of the puerarin derivatives provided by the present disclosure to inhibit the small intestinal lipid absorption, reduce the blood lipid level, and reduce the body weight, where FIG. 8A shows a chemical structural formula of puerarin derivative-1; FIG. 8B shows body weight curves of obese mouse models in the control group and the derivative-1 group; FIG. 8C shows the quantitative measurement results of blood lipid levels in the control group and the derivative-1 group; FIG. 8D shows the measurement results of jejunal TG levels in the control group and the derivative-1 group; FIG. 8E shows a chemical structural formula of puerarin derivative-2; FIG. 8F shows body weight curves of obese mouse models in the control group and the derivative-2 group; FIG. 8G shows the quantitative measurement results of blood lipid levels in the control group and the derivative-2 group; FIG. 8H shows the measurement results of jejunal TG levels in the control group and the derivative-2 group; FIG. 8I shows a chemical structural formula of puerarin derivative-3; FIG. 8J shows body weight curves of obese mouse models in the control group and the derivative-3 group; FIG. 8K shows the quantitative measurement results of blood lipid levels in the control group and the derivative-3 group; and FIG. 8L shows the measurement results of jejunal TG levels in the control group and the derivative-3 group.

### DETAILED DESCRIPTION OF THE EMBODIMENTS

The present disclosure provides a use of a GABA_{A} receptor as a drug target in the screening of a drug with one or more functions selected from the group consisting of functions (1) to (4): (1) reducing blood lipid; (2) treating obesity; (3) improving metabolic syndrome; and (4) inhibiting small intestinal lipid absorption. The GABA_{A} receptor is a chloride channel receptor in nerve cells, and the GABA_{A} receptor binds to γ-aminobutyric acid (namely, GABA) in a physiological state to promote the opening of a chloride channel, resulting in the hyperpolarization of nerve cells and the inhibition of nerve excitability.

The present disclosure also provides a use of a substance for increasing an expression level of a GABA_{A} receptor in the preparation of a drug with one or more functions selected from the group consisting of functions (1) to (4): (1) reducing blood lipid; (2) treating obesity; (3) improving metabolic syndrome; and (4) inhibiting small intestinal lipid absorption.

The present disclosure also provides a use of a substance for improving sensitivity of a GABA_{A} receptor or a chloride channel opening frequency, increasing a chloride influx, or causing nerve cell hyperpolarization in the preparation of a drug with one or more functions selected from the group consisting of functions (1) to (4): (1) reducing blood lipid; (2) treating obesity; (3) improving metabolic syndrome; and (4) inhibiting small intestinal lipid absorption.

The present disclosure also provides a use of a GABA_{A} receptor agonist (an agonist targeting the GABA_{A} receptor in the brainstem DMV) and/or allosteric modulator in the preparation of a drug with one or more functions selected from the group consisting of functions (1) to (4): (1) reducing blood lipid; (2) treating obesity; (3) improving metabolic syndrome; and (4) inhibiting small intestinal lipid absorption. Cryo-electron microscopy (Cryo-EM) analysis results show that puerarin can act on a site between α1 and γ2 subunits of the GABA_{A} receptor, and puerarin can bind to the GABA_{A} receptor to promote the opening of a chloride channel, thereby significantly increasing an inhibitory effect of γ-aminobutyric acid on neurons. Therefore, puerarin is an agonist for the GABA_{A} receptor in DMV.

In the present disclosure, the GABA_{A} receptor agonist includes puerarin and/or a derivative thereof, or a benzodiazepine; and the benzodiazepine includes one or more selected from the group consisting of flunitrazepam, diazepam, triazolam, and flumazenil.

The present disclosure also provides a use of a substance for inhibiting nerve excitability of the DMV in the preparation of a drug with one or more functions selected from the group consisting of functions (1) to (4): (1) reducing blood lipid; (2) treating obesity; (3) improving metabolic syndrome; and (4) inhibiting small intestinal lipid absorption. The DMV in the mammalian brainstem innervates the visceral movement and regulates the digestion and absorption, and the GABA_{A} receptor is expressed and enriched in the DMV. Previous studies have proved that the GABA_{A} receptor can be used as a drug target for sedation, anti-anxiety, anti-convulsion, and anti-depression; and the use of the GABA_{A} receptor in the DMV as a drug target for treating obesity, reducing blood lipid, improving metabolism, and inhibiting small intestinal lipid absorption is disclosed for the first time in the present disclosure. In the present disclosure, the excitability of DMV in mice is inhibited through chemical genetics, and then metabolic indexes such as body weight curve, blood lipid level, and small intestinal absorption are analyzed for the mice; and analysis results show that the inhibition of the DMV excitability can inhibit the small intestinal lipid absorption.

In the present disclosure, the substance for inhibiting nerve excitability of the DMV includes a substance for increasing an expression level of a GABA_{A} receptor or a substance for improving sensitivity of a GABA_{A} receptor or a chloride channel opening frequency, increasing a chloride influx, or causing nerve cell hyperpolarization.

The present disclosure also provides a use of puerarin and/or a derivative thereof in the preparation of a drug with one or more functions selected from the group consisting of functions (1) to (4): (1) reducing blood lipid; (2) treating obesity; (3) improving metabolic syndrome; and (4) inhibiting small intestinal lipid absorption. Puerarin is an isoflavone compound, which is the main active ingredient of the traditional Chinese medicine (TCM) Radix Puerariae and has a molecular formula I:

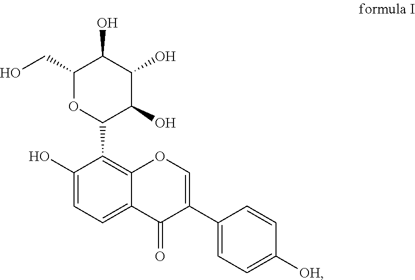

In the present disclosure, through electrophysiological analysis of brain slices, it is found that puerarin (and a derivative thereof) can inhibit the excitability of the DMV so as to inhibit the lipid absorption in a small intestine through “DMV-GABA_{A} receptor-vagus-small intestinal nutrient absorption” (the GABA_{A} receptor is highly expressed in the DMV with relatively high specificity; in a normal physiological state, the endogenous neurotransmitter γ-aminobutyric acid can bind to the GABA_{A} receptor to inhibit the excitability of neurons; and after binding to the GABA_{A} receptor, puerarin can increase the response of the GABA_{A} receptor to γ-aminobutyric acid, that is, in the case of receiving a small amount of γ-aminobutyric acid, the DMV can produce an improved inhibitory effect on the small intestine under the action of puerarin). Animal research results show that puerarin (and a derivative thereof) can inhibit the absorption of lipid nutrients in a small intestine through the vagus and promote the excretion of unabsorbed lipids through feces, thereby achieving the effects of reducing blood lipid, reducing body weight, and treating obesity.

A puerarin probe is used to prove that puerarin is enriched in the DMV and presents colocalization staining with the brainstem GABA_{A} receptor. With the help of cryo-EM, it is discovered in the present disclosure that puerarin acts on a drug target of the brainstem GABA_{A} receptor and binds to a site between α1 and γ2 subunits of the GABA_{A} receptor, which inhibits the nerve excitability of the DMV by regulating a chloride channel (that is, puerarin can bind to the GABA_{A} receptor to promote the opening of the chloride channel, thereby significantly increasing the inhibitory effect of γ-aminobutyric acid on neurons).

The present disclosure also provides a use of a gene Gabra1 and/or a gene Gabrg2 as a drug target in the screening of a drug with one or more functions selected from the group consisting of functions (1) to (4): (1) reducing blood lipid; (2) treating obesity; (3) improving metabolic syndrome; and (4) inhibiting small intestinal lipid absorption. It is proved through knockdown of Gabra1 (a gene for the α1 subunit of the GABA_{A} receptor) or Gabrg2 (a gene for the γ2 subunit of the GABA_{A} receptor) by shRNA that puerarin inhibits the small intestinal lipid absorption through the GABA_{A} receptor. Specifically in the present disclosure, by tissue-specific knockdown of the expression of the α1 subunit of the GABA_{A} receptor in the DMV with a Gabra1 shRNA virus (rAAV-DIO-Gabra1-shRNA, Brain VTA, China), and by illustrating the role of the GABA_{A} receptor in a process of puerarin to inhibit small intestinal lipid absorption, and reduce blood lipid and body weight in an obese mouse model, the brain-intestine regulatory pathway of “DMV-GABA_{A} receptor-vagus-small intestinal lipid absorption” is elucidated. Similarly, the present disclosure also proves through a Gabrg2 shRNA virus rAAV-DIO-Gabrg2-shRNA (BrainVTA, China) that the tissue-specific knockdown of Gabrg2 in DMV can block the inhibitory effect of puerarin on vagus and intervene in the effects of small intestinal lipid absorption-inhibiting, weight-losing, and lipid-lowering.

In order to further illustrate the present disclosure, the use of a GABA_{A} receptor as a target in the screening of a drug for reducing blood lipid, treating obesity, and/or improving metabolism provided by the present disclosure is described in detail below in conjunction with accompanying drawings and examples, but these examples should not be understood as limiting the claimed scope of the present disclosure.

### Example 1

### 1. Establishment and Detection of an Experimental Animal Model

### (1) Establishment of an Obese Animal Model

The C57BL/6J mice were used as experimental mice in the present disclosure. The experimental mice each were raised in an SPF-level environment at 22° C. to 24° C. with a 12:12 circadian rhythm, and the experimental mice each were subjected to anaesthesia, analgesia, surgery, intraperitoneal injection, and other experimental operations in accordance with the relevant provisions of animal ethics.

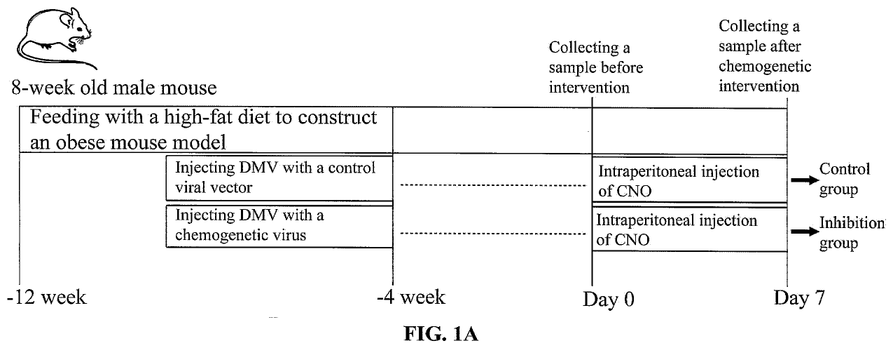

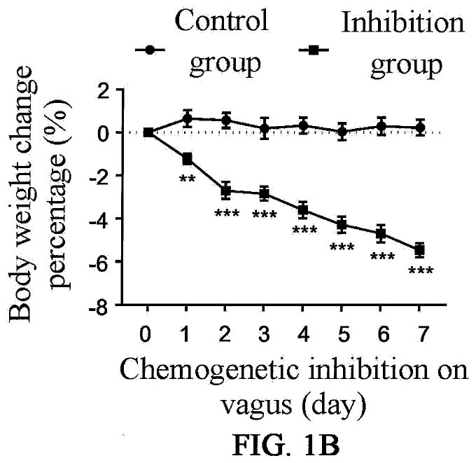

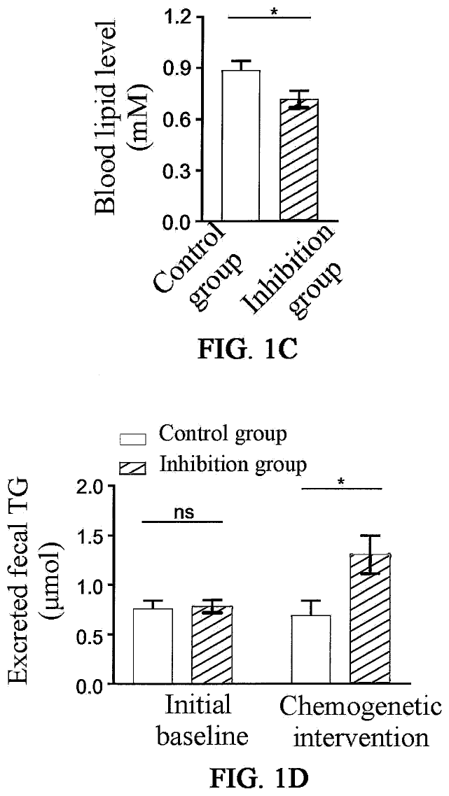

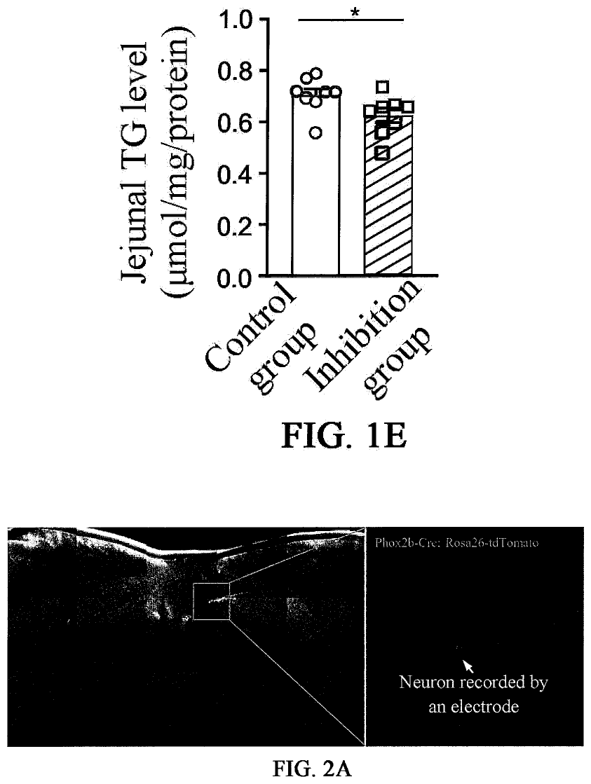

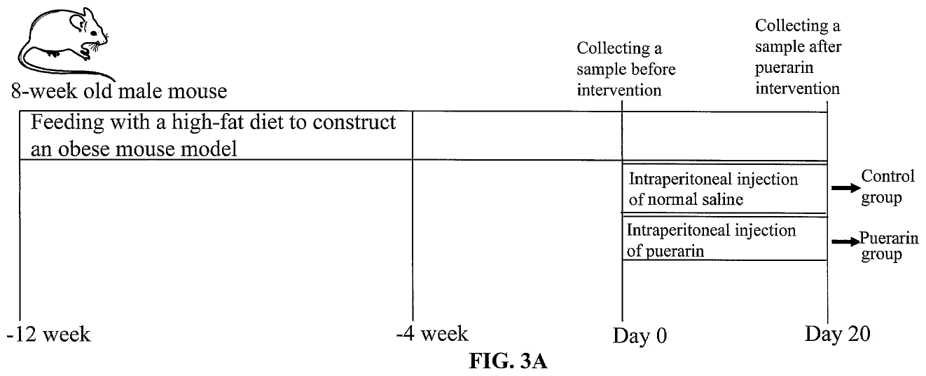

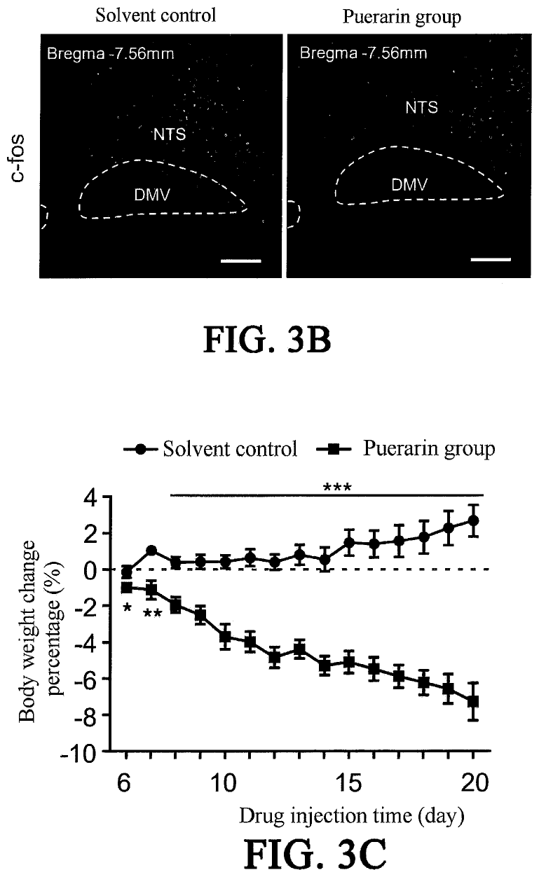

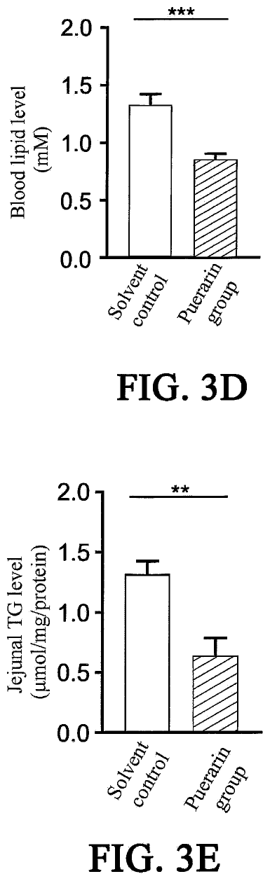

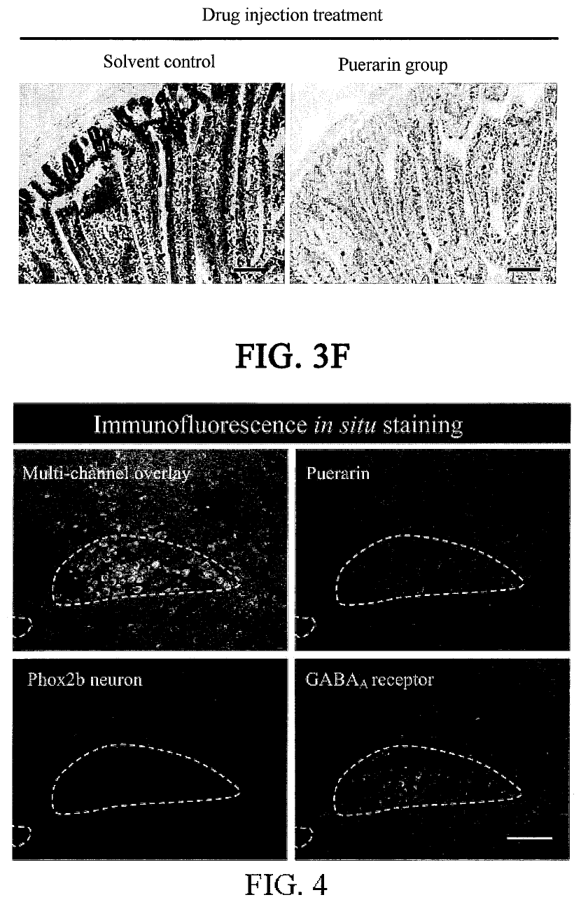

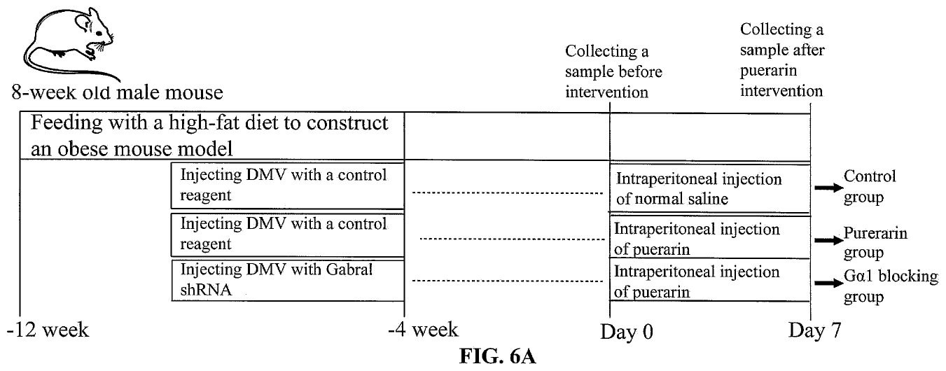

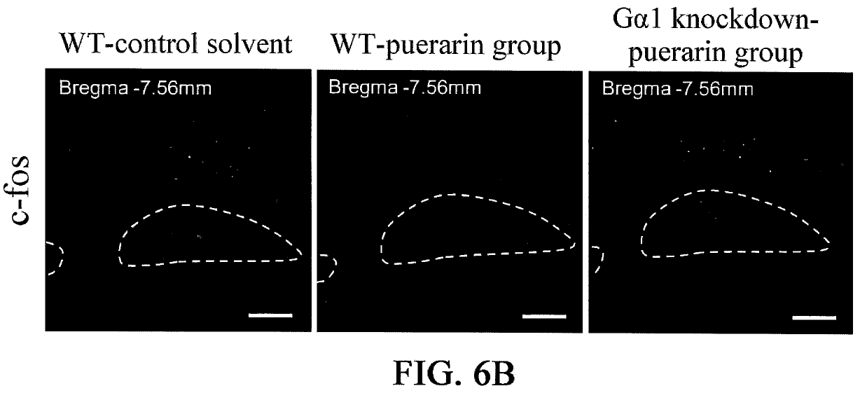

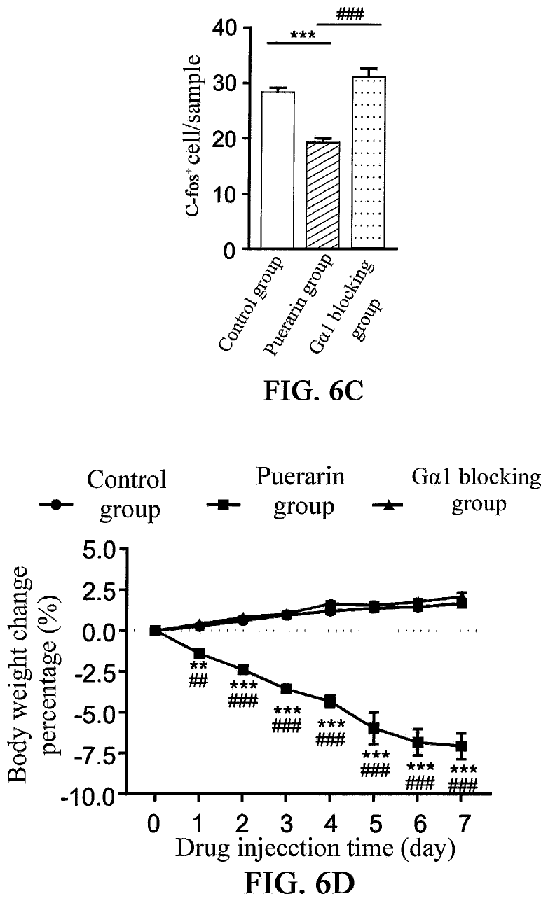

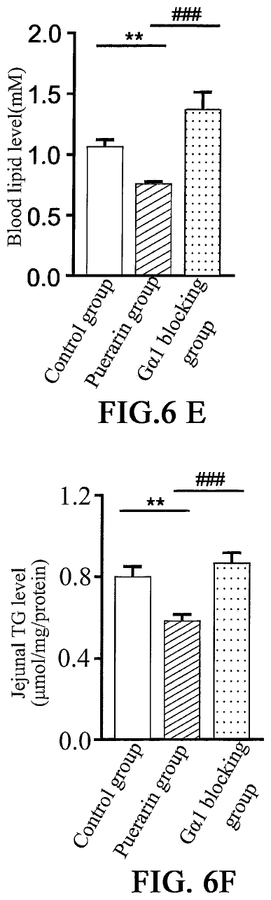

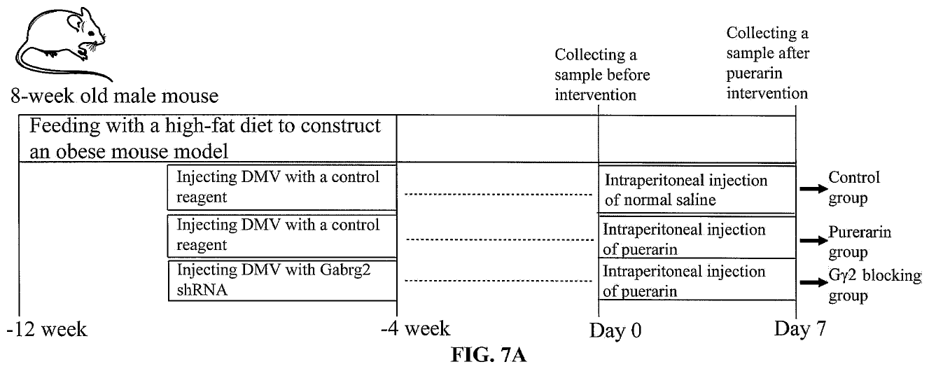

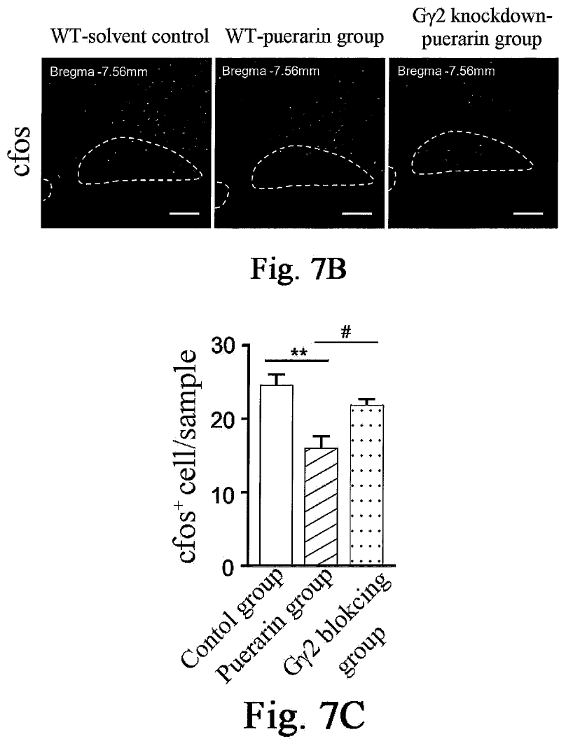

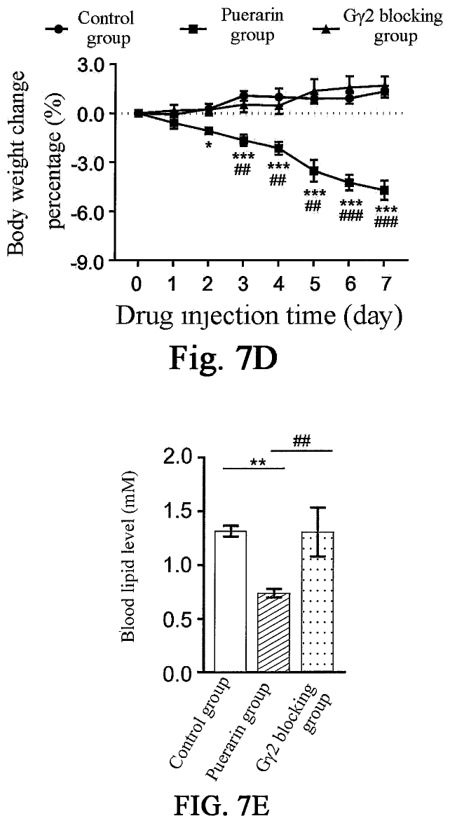

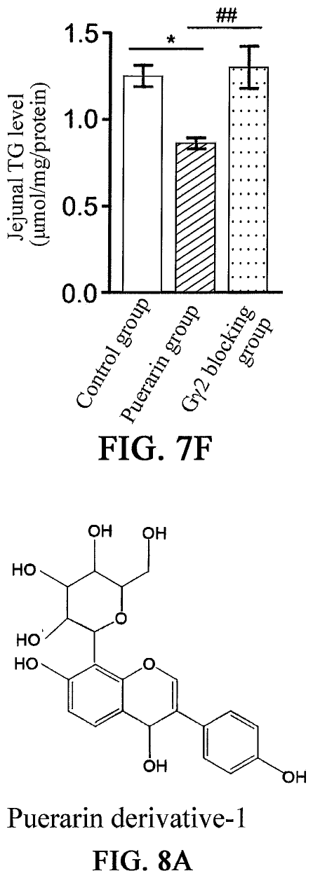

In the present disclosure, the mice were fed with a high-fat diet in which a mass percentage of fat was 60% for 12 weeks, and when a body weight reached 35 g or more, animal models of alimentary obesity were successfully constructed. At a formal stage of the experiment (starting on day 0 in FIG. 1A, FIG. 3A, FIG. 6A, and FIG. 7A), the mice were raised in single cages, during which a daily food intake of each mouse model was accurately recorded, the feces was collected before and after drug intervention, and a daily body weight curve was recorded.

### (2) Chemogenetic Virus Injection

The chemogenetic approach designer receptors exclusively activated by designer drugs (DREADDs) is a research method that controls neuronal excitability in a specific brain region in an animal model, where a downstream signaling pathway of a specific exogenous ligand clozapine N-oxide (CNO) is activated through an adeno-associated virus (AAV) modified by a G protein-coupled receptor (GPCR) to cause a change in neuronal excitability. In the present disclosure, with DMV as a breakthrough point, the inhibitory chemogenetic virus (rAAV-Efla-DIO-hM4D(Gi)-mCherry-WPRE-pA, purchased from BrainVTA, China) and the control virus (rAAV-Efla-DIO-mCherry-WPRE-pA, purchased from BrainVTA, China) were used to construct DMV-intervened mouse models. Specific experimental operations were as follows:

The mice were deeply anesthetized with pentobarbital sodium, fixed on a brain solid positioner, and cut to expose a cranial surface; the DMV (DMV coordinates: −7.10 mm, ±0.2 mm, and −4.50 mm) was localized according to Bregma points, the cranium was drilled, and 300 nL of a virus was injected by a microsampler at each side with injection for 5 min and stabilization for 5 min; after the injection was completed, the cranial surface was smeared with a chlortetracycline ointment for anti-inflammation, and then a surface of a wound was sutured; and the mice were placed on a heating pad at 37° C. to recover for 1 h, and then put back to cages for waking up. In the chemogenetic inhibition group and the control group, the inhibitory chemogenetic virus (rAAV-Efla-DIO-hM4D(Gi)-mCherry-WPRE-pA, BrainVTA, China) and the control virus (rAAV-Efla-DIO-mCherry-WPRE-pA, BrainVTA, China) were injected into the DMV, respectively, and the other operations were completely consistent.

Four weeks after recovery from surgery, mice in the chemogenetic inhibition group and the control group each were intraperitoneally injected with CNO (Catalog No. BML-NS105, Enzo Life Sciences, USA) at a dose of 1.0 mg/kg BW every day to inhibit the neuronal activity of the DMV, and the daily food intake and body weight were measured and the feces was collected for the mice. The mice were sacrificed after being continuously injected with CNO for 7 d to inhibit the DMV, mouse tissue samples were collected, and physiological and metabolic indexes were detected and analyzed.

### 2. Detection and Analysis of Physiological Metabolic Indexes

The physiological and metabolic indexes involved in the present disclosure included: triglyceride (TG) and non-esterified fatty acid (NEFA) contents in samples such as plasma, intestinal tissue, and feces.

A method for determining TG in a sample was as follows: 20 mg to 30 mg of a sample was first weighed, a 5% NP40-containing lysis buffer was added, and a resulting mixture was homogenated and incubated in a metal bath at 95° C. for 5 min, which was repeated twice. A resulting homogenate was cooled to room temperature and then centrifuged at 15,000 rpm for 2 min to 3 min, and a resulting supernatant was pipetted for determination. A Biovision kit was used to determine TG. 2 μl of the supernatant was added to each well, and after a chromogenic reaction was completed, a value of each well was determined by a microplate reader. TG in the small intestinal tissue was quantified through a standard protein concentration; and a total amount of lipids excreted with the feces was calculated based on a measured value of the feces sample and a total mass of the sample at 24 h.

NEFA in a sample was extracted through isopropanol/chloroform/Triton-100 extraction, dried in an oven at 50° C., vacuumed by a vacuum pump for 30 min, and then dissolved in a 5% NP-40 solution. Subsequently, the difference between quantification values of the microplate reader before and after the sample was added to the Wako kit (catalog No. 294-63601, WAKO, Japan) was determined to quantify NEFA in the sample.

Based on the above physiological index detection and analysis embodiment, the body weight curve, fecal lipid, intestinal lipid, blood lipid, and other lipid metabolism-associated physiological indexes of the animal model were mainly detected in the present disclosure.

FIGS. 1A-1E show a regulation effect of the DMV on the small intestinal lipid absorption that is demonstrated by a chemogenetic strategy. FIG. 1A is an experimental flow chart of the chemogenetic strategy: 8-week-old male mice were taken to construct mouse models of alimentary obesity. The mice were fed with a high-fat feed for 8 weeks and then randomly divided into two groups, and the two groups were injected with the inhibitory chemogenetic virus and the control virus respectively into the DMV to construct an inhibition animal model group and a control animal model group. The mice were further fed with a high-fat feed for 4 weeks, and when mice each had a body weight of 35 g or more, the body weight was recorded and the fecal sample was collected as initial baselines, and this day was set as day 0. From day 1, the mice were intraperitoneally injected with CNO to activate the DMV through the chemogenetic strategy, where CNO was injected once a day continuously for 7 d and a daily body weight change was recorded. The mice were sacrificed 2 h after the CNO injection on day 7, the blood, feces, small intestinal tissue, and the like were collected, and the lipid in each sample was quantitatively determined, and the experimental results were obtained as shown in FIG. 1 B to FIG. 1E.

After the chemogenetic inhibition of neurons in the DMV (namely, after the intraperitoneal injection of CNO), the body weight of each obese mice in the inhibition group gradually decreased; and after the neurons in the DMV were inhibited for 7 d, the body weight of each obese mice in the inhibition group was significantly lower than that in the control group (FIG. 1B, Table 1), and the blood lipid level was significantly reduced (FIG. 1C, Table 2). As shown in FIG. 1D and Table 3, comparison between results before and after the intervention of the chemogenetic strategy (namely, before the CNO injection, and 7 d after the CNO injection) showed that a large amount of unabsorbed TG can be excreted through feces in the inhibition group; and after the inhibition on DMV by the chemogenetic strategy, the lipid nutrients such as TG absorbed into the small intestine through a jejunum segment were significantly reduced (FIG. 1E, Table 4). The above experimental results showed that the inhibition on the DMV significantly reduced the small intestinal lipid absorption, lower the blood lipid level, and reduce the body weight.

TABLE 1
Body weight curve change percentage (%)

| | Day | Day | Day | Day |
|---|---|---|---|---|
| | 0 | 1 | 2 | 3 |
| Control | 0.00% | 0.27% | 0.54% | 0.54% |
| group-01 | | | | |
| Control | 0.00% | 1.95% | 1.68% | 1.12% |
| group-02 | | | | |
| Control | 0.00% | 1.05% | 0.00% | −1.62% |
| group-03 | | | | |
| Control | 0.00% | 0.27% | 1.08% | 0.81% |
| group-04 | | | | |
| Control | 0.00% | 2.49% | 2.25% | 2.74% |
| group-05 | | | | |
| Control | 0.00% | −0.26% | −0.26% | −0.78% |
| group-06 | | | | |
| Control | 0.00% | −0.76% | −0.50% | −0.76% |
| group-07 | | | | |
| Control | 0.00% | 0.26% | −0.27% | −0.53% |
| group-08 | | | | |
| Mean | 0.00% | 0.66% | 0.56% | 0.19% |
| Inhibition | 0.00% | −0.52% | −1.58% | −2.39% |
| group-01 | | | | |
| Inhibition | 0.00% | 0.00% | −2.59% | −2.59% |
| group-02 | | | | |
| Inhibition | 0.00% | −1.64% | −3.33% | −2.76% |
| group-03 | | | | |
| Inhibition | 0.00% | −2.24% | −5.19% | −1.39% |
| group-04 | | | | |
| Inhibition | 0.00% | −1.34% | −2.17% | −2.72% |
| group-05 | | | | |
| Inhibition | 0.00% | −1.15% | −1.73% | −2.62% |
| group-06 | | | | |
| Inhibition | 0.00% | −1.31% | −2.39% | −4.04% |
| group-07 | | | | |
| Inhibition | 0.00% | −1.53% | −2.58% | −4.20% |
| group-08 | | | | |
| Mean | 0.00% | −1.22% | −2.70% | −2.84% |
| t-test | 0 ns | 0.00109896 ** | 3.37553E−05 *** | 6.74263E−06 *** |

| | Day | Day | Day | Day |
|---|---|---|---|---|
| | 4 | 5 | 6 | 7 |
| Control | 0.54% | 0.54% | 0.81% | 0.81% |
| group-01 | | | | |
| Control | 1.12% | 1.40% | 0.85% | 1.12% |
| group-02 | | | | |
| Control | −1.08% | −1.62% | −1.35% | −0.80% |
| group-03 | | | | |
| Control | 1.08% | 1.60% | 1.34% | 0.54% |
| group-04 | | | | |
| Control | 2.01% | −0.77% | 2.01% | 2.01% |
| group-05 | | | | |
| Control | −0.52% | 0.00% | −0.52% | −0.78% |
| group-06 | | | | |
| Control | −0.50% | −0.25% | −0.50% | −0.50% |
| group-07 | | | | |
| Control | 0.00% | −0.53% | −0.27% | −0.53% |
| group-08 | | | | |
| Mean | 0.33% | 0.05% | 0.30% | 0.23% |
| Inhibition | −2.94% | −3.22% | −3.77% | −4.34% |
| group-01 | | | | |
| Inhibition | −3.19% | −3.79% | −4.40% | −5.33% |
| group-02 | | | | |
| Inhibition | −3.91% | −5.38% | −4.49% | −5.08% |
| group-03 | | | | |
| Inhibition | −1.96% | −2.82% | −3.11% | −4.89% |
| group-04 | | | | |
| Inhibition | −3.01% | −4.14% | −4.14% | 4.72% |
| group-05 | | | | |
| Inhibition | −3.83% | −3.83% | −5.39% | −6.02% |
| group-06 | | | | |
| Inhibition | −4.32% | −5.46% | −6.63% | −6.63% |
| group-07 | | | | |
| Inhibition | −5.59% | −5.59% | −5.59% | −6.72% |
| group-08 | | | | |
| Mean | −3.59% | −4.28% | −4.69% | −5.47% |
| t-test | 1.90462E−06 *** | 2.99658E−07 *** | 1.49375E−07 *** | 7.33759E−09 *** |

TABLE 2
Blood lipid level (mM)

| | |
|---|---|
| Control group | Inhibition group |

| | | | |
|---|---|---|---|
| Control group-01 | 0.6700 | Inhibition group-01 | 0.4645 |
| Control group-02 | 1.0005 | Inhibition group-02 | 0.8754 |
| Control group-03 | 0.9111 | Inhibition group-03 | 0.6342 |
| Control group-04 | 0.9826 | Inhibition group-04 | 0.7146 |
| Control group-05 | 1.0987 | Inhibition group-05 | 0.7414 |
| Control group-06 | 0.6878 | Inhibition group-06 | 0.7414 |
| Control group-07 | 0.9290 | Inhibition group-07 | 0.9111 |
| Control group-08 | 0.8218 | Inhibition group-08 | 0.6521 |
| Mean | 0.8877 | Mean | 0.7169 |

TABLE 3
Excreted TG (μmol) in feces

| Day 0 (control group) | | Day 0 (inhibition group) | | Day 7 (inhibition group) | | Day 7 (inhibition group) | |
|---|---|---|---|---|---|---|---|
| Control | 0.6908 | Inhibition | 0.4866 | Control | 0.3269 | Inhibition | 1.8286 |
| group-01 | | group-01 | | group-01 | | group-01 | |
| Control | 0.3186 | Inhibition | 0.9129 | Control | 0.5096 | Inhibition | 0.9268 |
| group-02 | | group-02 | | group-02 | | group-02 | |
| Control | 0.8951 | Inhibition | 0.7756 | Control | 0.4966 | Inhibition | 1.5860 |
| group-03 | | group-03 | | group-03 | | group-03 | |
| Control | 0.5996 | Inhibition | 0.6665 | Control | 0.3774 | Inhibition | 1.9107 |
| group-04 | | group-04 | | group-04 | | group-04 | |
| Control | 0.8481 | Inhibition | 0.7774 | Control | 1.4592 | Inhibition | 1.8898 |
| group-05 | | group-05 | | group-05 | | group-05 | |
| Control | 0.9437 | Inhibition | 0.8718 | Control | 1.0478 | Inhibition | 0.8193 |
| group-06 | | group-06 | | group-06 | | group-06 | |
| Control | 0.8235 | Inhibition | 0.7016 | Control | 0.3741 | Inhibition | 0.7688 |
| group-07 | | group-07 | | group-07 | | group-07 | |
| Control | 0.9920 | Inhibition | 1.0872 | Control | 0.9836 | Inhibition | 0.7149 |
| group-08 | | group-08 | | group-08 | | group-08 | |
| Mean | 0.7639 | | 0.7849 | | 0.6969 | | 1.3056 |

TABLE 4
Jejunal TG level

| | |
|---|---|
| Control group | Inhibition group |

| | | | |
|---|---|---|---|
| Control group-01 | 0.6794 | Inhibition group-01 | 0.5607 |
| Control group-02 | 0.5593 | Inhibition group-02 | 0.6672 |
| Control group-03 | 0.6969 | Inhibition group-03 | 0.6428 |
| Control group-04 | 0.7194 | Inhibition group-04 | 0.4794 |
| Control group-05 | 0.7121 | Inhibition group-05 | 0.6581 |
| Control group-06 | 0.7892 | Inhibition group-06 | 0.7370 |
| Control group-07 | 0.7699 | Inhibition group-07 | 0.6580 |
| Control group-08 | 0.7154 | Inhibition group-08 | 0.5973 |
| Mean | 0.7052 | Mean | 0.6251 |

The results of puerarin to inhibit the small intestinal lipid absorption and reduce the body weight in obese mice were shown in FIG. 3. In the present disclosure, the above physiological indexes were also detected and analyzed to clarify the improvement effect of puerarin on the body weight curve, fecal lipid, intestinal lipid, blood lipid, and other lipid metabolism-associated physiological indexes of obese mice. FIG. 3A is a schematic diagram illustrating a process of intraperitoneal injection of puerarin into the obese mouse model: 8-week-old male mice were taken and fed with a high-fat feed for 12 weeks until the mice had a body weight of 35 g or more, then the obese mouse models were randomly divided into two groups, and the two groups were intraperitoneally injected with normal saline (NS) and a puerarin solution respectively to construct a control animal model group and a puerarin animal model group. Before the intraperitoneal injection of puerarin, a feces sample was collected and a body weight was recorded as initial baselines, and this day was set as day 0. From day 1, NS and the puerarin solution were intraperitoneally injected once a day continuously for 20 d, during which a daily body weight change was recorded. 2 h after the puerarin injection on day 20, the mice were sacrificed, the blood, feces, small intestine, brain, and the like were collected, the physiological indexes such as blood lipid and jejunal TG were quantitatively determined, and the morphological results of the brainstem DMV and small intestine were directly determined through tissue slices (including brain slice c-fos immunofluorescence staining test and small intestine oil red O staining test).

The brain slice c-fos immunofluorescence staining results are shown in FIG. 3B, where the number of fluorescence signals represents an excitability degree of neurons and the results are representative brain slice c-fos staining results for the puerarin group and the control group. A number of c-fos fluorescence signals in the DMV brain region in the puerarin group was smaller than that in the control group, indicating that the neuronal excitability of the DMV was inhibited by puerarin. During puerarin injection, the body weight curves (FIG. 3C, Table 5) of the puerarin experimental group and the control group were recorded, and the blood lipid level (FIG. 3D, Table 6) was measured; and the jejunal lipid absorption ability was detected by the Biovision kit (FIG. 3E, Table 7). FIG. 3F shows representative oil red O staining results of the puerarin group and the control group, which visually present a total amount of lipids absorbed in the small intestine. The above experimental results showed that puerarin inhibited the excitability of neurons in the DMV, and the small intestinal lipid absorption was inhibited, the blood lipid level was reduced, and the body weight was gradually reduced with the injection of puerarin, thereby achieving the effects of weight loss and lipid reduction.

TABLE 5
Body weight curve change percentage (%)

| | Day | Day | Day | Day | Day | Day | Day | Day |
|---|---|---|---|---|---|---|---|---|
| | 6 | 7 | 8 | 9 | 10 | 11 | 12 | 13 |
| Solvent | −0.80% | −0.27% | −0.53% | −0.27% | −0.53% | −0.27% | 0.00% | 0.26% |
| control-01 | | | | | | | | |
| Solvent | 0.00% | 1.96% | 0.85% | 0.28% | 1.13% | 0.57% | 0.00% | 1.69% |
| control-02 | | | | | | | | |
| Solvent | 0.27% | 0.27% | −1.10% | −1.10% | 0.00% | −1.10% | 0.00% | −1.38% |
| control-03 | | | | | | | | |
| Solvent | −0.27% | 2.14% | 1.61% | 1.88% | 1.88% | 3.17% | 2.14% | 3.68% |
| control-04 | | | | | | | | |
| Solvent | 0.00% | 1.59% | 1.06% | 1.59% | 1.59% | 1.59% | 1.06% | 1.85% |
| control-05 | | | | | | | | |
| Solvent | −1.74% | 1.68% | −0.57% | 0.00% | 0.00% | 0.00% | −0.29% | 0.00% |
| control-06 | | | | | | | | |
| Solvent | −0.55% | 0.82% | 0.27% | −0.28% | 0.00% | 0.00% | −1.11% | −0.55% |
| control-07 | | | | | | | | |
| Solvent | −1.09% | 0.80% | −0.27% | −0.81% | −0.27% | −0.27% | 0.00% | 0.53% |
| control-08 | | | | | | | | |
| Solvent | 1.76% | 0.26% | 1.01% | 0.51% | −1.30% | −0.26% | −1.03% | −1.03% |
| control-09 | | | | | | | | |
| Solvent | 1.03% | 1.28% | 1.53% | 2.53% | 1.78% | 3.02% | 3.26% | 3.02% |
| control-10 | | | | | | | | |
| Mean | −0.14% | 1.05% | 0.39% | 0.43% | 0.43% | 0.65% | 0.40% | 0.81% |
| Puerarin | −1.08% | −0.81% | −1.36% | −1.08% | −2.19% | −3.32% | −3.32% | −4.19% |
| group-01 | | | | | | | | |
| Puerarin | 0.00% | 2.41% | 0.00% | −1.11% | −1.11% | −3.11% | −3.99% | −2.24% |
| group-02 | | | | | | | | |
| Puerarin | 0.00% | 0.00% | −0.80% | −0.80% | −1.62% | −2.17% | −3.57% | −3.29% |
| group-03 | | | | | | | | |
| Puerarin | −0.79% | −0.26% | −0.79% | −0.79% | −2.13% | −2.95% | −4.35% | −5.21% |
| group-04 | | | | | | | | |
| Puerarin | −0.57% | −1.74% | −2.93% | −3.54% | −4.15% | −4.78% | −6.04% | −4.46% |
| group-05 | | | | | | | | |
| Puerarin | −0.55% | −0.83% | −0.83% | −1.39% | −1.67% | −1.39% | −1.67% | −2.24% |
| group-06 | | | | | | | | |
| Puerarin | −2.01% | −3.04% | −3.83% | −4.36% | −6.54% | −7.11% | −7.39% | −6.54% |
| group-07 | | | | | | | | |
| Puerarin | −1.82% | −2.89% | −3.71% | −4.55% | −6.54% | −6.25% | −6.83% | −7.12% |
| group-08 | | | | | | | | |
| Puerarin | −1.41% | −1.69% | −2.56% | −3.45% | −5.57% | −4.35% | −6.51% | −4.35% |
| group-09 | | | | | | | | |
| Puerarin | −1.70% | −2.37% | −2.65% | −4.02% | −5.43% | −4.30% | −4.72% | −4.16% |
| group-10 | | | | | | | | |
| Mean | −0.99% | −1.12% | −1.95% | −2.51% | −3.70% | −3.97% | −4.84% | −4.38% |
| t-test | 0.044344 * | 0.001319 ** | 0.000316 *** | 0.000194 *** | 4.39E−05 *** | 5.35E−06 *** | 9.19E−07 *** | 1.55E−06 *** |

| | Day | Day | Day | Day | Day | Day | Day |
|---|---|---|---|---|---|---|---|
| | 14 | 15 | 16 | 17 | 18 | 19 | 20 |
| Solvent | 0.00% | −0.27% | −0.27% | 0.00% | 0.79% | 1.05% | 0.79% |
| control-01 | | | | | | | |
| Solvent | 2.23% | 4.11% | 3.05% | 2.23% | 3.05% | 4.37% | 4.37% |
| control-02 | | | | | | | |
| Solvent | −2.51% | −1.10% | −1.66% | −2.80% | −2.51% | −1.94% | 0.00% |
| control-03 | | | | | | | |
| Solvent | 3.68% | 4.44% | 5.43% | 5.67% | 6.63% | 7.11% | 6.87% |
| control-04 | | | | | | | |
| Solvent | 1.06% | 2.36% | 1.33% | 2.11% | 2.11% | 2.62% | 3.88% |
| control-05 | | | | | | | |
| Solvent | 0.00% | 0.28% | 0.57% | 0.00% | 0.00% | 0.57% | 1.40% |
| control-06 | | | | | | | |
| Solvent | −1.40% | −0.83% | −0.83% | −0.28% | −0.55% | 0.00% | 0.00% |
| control-07 | | | | | | | |
| Solvent | 0.00% | 1.85% | 1.85% | 2.36% | 2.62% | 3.13% | 3.88% |
| control-08 | | | | | | | |
| Solvent | −1.03% | −0.51% | 0.00% | 0.26% | 0.00% | −0.51% | −0.77% |
| control-09 | | | | | | | |
| Solvent | 3.50% | 4.46% | 4.46% | 6.08% | 5.62% | 6.31% | 6.31% |
| control-10 | | | | | | | |
| Mean | 0.55% | 1.48% | 1.39% | 1.56% | 1.77% | 2.27% | 2.67% |
| Puerarin | −4.48% | −3.61% | −4.19% | −4.19% | −4.19% | −3.90% | −4.48% |
| group-01 | | | | | | | |
| Puerarin | −4.89% | −3.11% | −3.99% | −6.10% | −7.67% | −6.10% | −7.35% |
| group-02 | | | | | | | |
| Puerarin | −4.14% | −4.14% | −5.01% | −4.72% | −4.43% | −5.31% | −5.31% |
| group-03 | | | | | | | |
| Puerarin | −5.49% | −6.37% | −6.37% | −5.79% | −6.67% | −7.56% | −7.56% |
| group-04 | | | | | | | |
| Puerarin | −6.04% | −3.85% | −3.85% | −5.41% | −5.09% | −4.46% | −4.78% |
| group-05 | | | | | | | |
| Puerarin | −2.24% | −2.53% | −2.82% | −3.40% | −3.99% | −4.58% | −4.29% |
| group-06 | | | | | | | |
| Puerarin | −7.11% | −8.24% | −9.12% | −9.12% | −9.70% | −10.90% | −14.01% |
| group-07 | | | | | | | |
| Puerarin | −8.31% | −7.71% | −8.31% | −9.22% | −9.52% | −11.08% | −11.71% |
| group-08 | | | | | | | |
| Puerarin | −5.26% | −5.88% | −5.26% | −4.65% | −4.96% | −5.88% | −5.88% |
| group-09 | | | | | | | |
| Puerarin | −4.86% | −5.58% | −5.87% | −6.16% | −6.01% | −6.01% | −7.48% |
| group-10 | | | | | | | |
| Mean | −5.28% | −5.10% | −5.48% | −5.87% | −6.22% | −6.58% | −7.28% |
| t-test | 1.6E−06 *** | 1.66E−06 *** | 1.29E−06 *** | 1.55E−06 *** | 1.16E−06 *** | 1.19E−06 *** | 6.98E−07 *** |

TABLE 6
Blood lipid level (mM)

| | |
|---|---|
| Solvent control | Puerarin group |

| | | | |
|---|---|---|---|
| Control group-01 | 1.6951 | Puerarin-01 | 0.8684 |
| Control group-02 | 1.6795 | Puerarin-02 | 0.6680 |
| Control group-03 | 1.5578 | Puerarin-03 | 0.7570 |
| Control group-04 | 1.5030 | Puerarin-04 | 0.9574 |
| Control group-05 | 1.3471 | Puerarin-05 | 0.6234 |
| Control group-06 | 1.3478 | Puerarin-06 | 1.1912 |
| Control group-07 | 0.9797 | Puerarin-07 | 0.8238 |
| Control group-08 | 0.8350 | Puerarin-08 | 0.7682 |
| Control group-09 | 1.0465 | Puerarin-09 | 0.9797 |
| Control group-10 | 1.2914 | Puerarin-10 | 0.8795 |
| Mean | 1.3283 | Mean | 0.8517 |

TABLE 7
Jejunal TG level (μmol/mg/protein)

| | |
|---|---|
| Solvent control | Puerarin group |

| | | | |
|---|---|---|---|
| Control group-01 | 1.6066 | Puerarin-01 | 1.0144 |
| Control group-02 | 1.5105 | Puerarin-02 | 0.2884 |
| Control group-03 | 1.0551 | Puerarin-03 | 0.2284 |
| Control group-04 | 1.4495 | Puerarin-04 | 1.4782 |
| Control group-05 | 1.0600 | Puerarin-05 | 1.2063 |
| Control group-06 | 1.1607 | Puerarin-06 | 0.2454 |
| Control group-07 | 1.1838 | Puerarin-07 | 0.3711 |
| Control group-08 | 0.7658 | Puerarin-08 | 0.8886 |
| Control group-09 | 1.9751 | Puerarin-09 | 0.1703 |
| Control group-10 | 1.4240 | Puerarin-10 | 0.4879 |
| Mean | 1.3191 | Mean | 0.6379 |

### 3. Electrophysiological Experiment on Brain Slices

Obese mouse models fed with a high-fat feed for 12 weeks were taken and myocardially perfused with an artificial cerebrospinal fluid (ACSF), and 200 μm to 300 μm-thick brainstem tissue slices were prepared by vibration slicing; and brain slices were incubated in oxygen at 35° C. for 1 h and then transferred to a patch-clamp system for whole-cell patch-clamp recording. Recorded neurons were selected according to the cellular morphology in the DMV, and a green-fluorescent dye was added to a pipette solution to label cells. In the present disclosure, the baseline, puerarin incubation, and elution stages were continuously recorded in a current clamp mode to clarify the influence of the drug treatment on neurons in the DMV: A spontaneous action potential of neurons was recorded for 1 min to 3 min at the baseline stage; then the spontaneous action potential was continuously recorded, the incubation of puerarin (concentration: 10 mM) was started, and an electrophysiological signal at the drug treatment stage was recorded for 5 min to 30 min; and finally, an ACSF elution process was recorded.

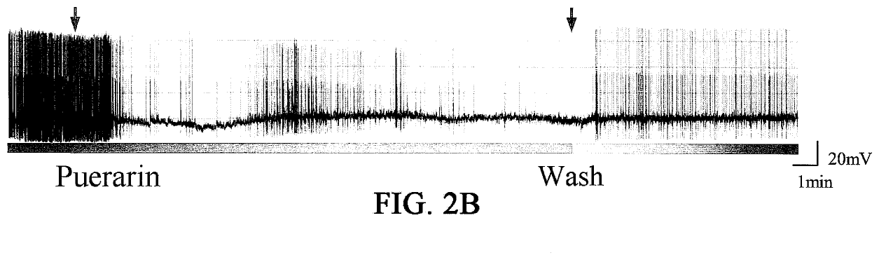

The inhibition results of puerarin on neurons in the DMV obtained through electrophysiological recording of brain slices are shown in FIG. 2.

FIG. 2A is a demonstration of an electrophysiological operation of a brainstem DMV brain slice. There was fluorescent dye (green) present, which was in the pipette solution of a recording electrode and could label neurons recorded by the electrode, indicating that nerve cells recorded in FIG. 2A (labeled with green fluorescence) are neurons of the DMV.

As shown in FIG. 2B, the electrophysiological results of brain slices showed that, during puerarin incubation, the action potential discharge frequency of neurons in the DMV was significantly reduced and the membrane potential was slightly lower than the initial baseline. In summary, puerarin may inhibit the electrophysiological activity of neurons in the DMV and the excitability of the neurons, thereby inhibiting the neurons in the DMV.

### 4. Cryo-EM Analysis of Drug Binding Site of Puerarin to Target Receptor

With reference to the method in the related literature (Dai J, Liang K, Zhao S, et al. Chemoproteomics reveals baicalin activates hepatic CPT1 to ameliorate diet-induced obesity and hepatic steatosis. Proc Natl Acad Sci USA. 2018; 115 (26): E5896-E5905), immunofluorescence staining was conducted for the puerarin and GABA_{A} receptor on a brain slice tissue, and the colocalization staining results of the puerarin and GABA_{A} receptor in the DMV are shown in FIG. 4. FIG. 4 shows the spatial specificity of binding of puerarin and the colocalization of puerarin with a target receptor, and it was observed that the puerarin probe was enriched in the DMV and showed prominent colocalization with staining of the GABA_{A} receptor, indicating that puerarin may target the GABA_{A} receptor in the DMV. According to the expression profiles of various genes in the publicly-available international database Allen Brain Atlas, the GABA_{A} receptor including α1β3γ2 (http://mouse.brain-map.org/gene/show/14170) is enriched in the DMV, and may be a molecular target of puerarin in the DMV.

In the present disclosure, with reference to the literature (Laverty, D. et al. Cryo-EM structure of the human alpha1beta3gamma2 GABA_{A} receptor in a lipid bilayer. Nature 565, 516-520), a stable 293S cell line in which the α1β3γ2 GABA_{A} receptor was enriched in a form of pentamer in the DMV was constructed, where the α1 subunit carried a Flag tag for protein purification; and about 2 L of a cell suspension was prepared. The cell suspension was subjected to ultrasonic cell disruption and then centrifuged by an ultracentrifuge to obtain a cell membrane pellet; then the cell membrane pellet was dissolved with a detergent for 2 h at 4° C., and then a membrane solution was acquired through high-speed centrifugation and co-incubated with an anti-flag resin to obtain a GABA_{A} receptor protein; and then a high-purity protein solution was acquired with a molecular sieve. The protein solution was concentrated to 2 mg/mL or higher and then incubated with puerarin for 30 min to obtain a GABA_{A} receptor protein sample binding to puerarin.

Preparation of cryo-EM samples: A porous carbon-film copper mesh that had undergone glow discharge was prepared, 3 μL of a drug-receptor binding solution was added dropwise to a surface of the copper mesh, the excess solution was removed, and then the copper mesh was quickly frozen in liquid ethane to a glass state and then stored in liquid nitrogen for cryo-EM analysis.

A 300 kV FEI Titan Krios cryo-electron microscope was used to acquire images of a total of 142,931 single-particle proteins, and the EM images of various visual angles were clustered and subjected to averaged 2D image construction, Fourier operation, and 3D model reconstruction to finally obtain a cryo-EM image with a resolution of 3.60 Å.

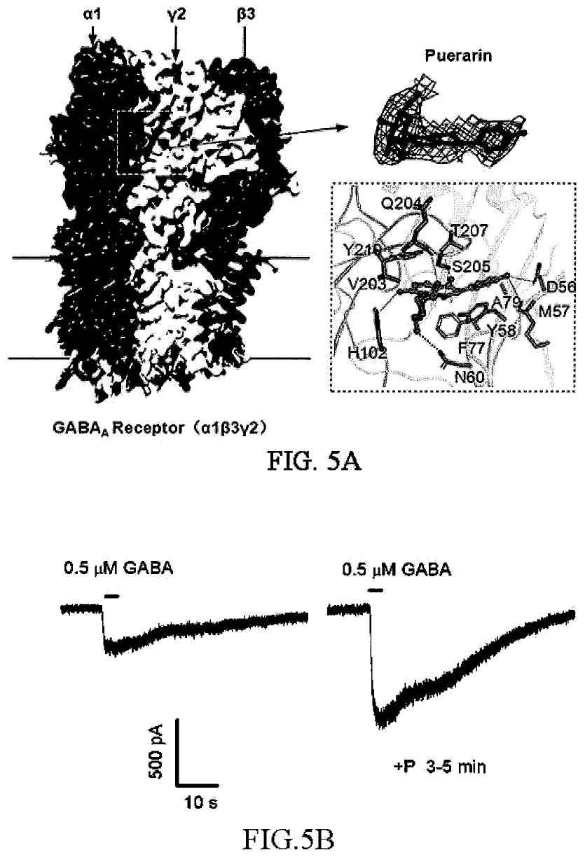

FIG. 5 shows the cryo-EM analysis results of the drug binding site of puerarin to the GABA_{A} receptor. FIG. 5A is a cryo-EM structural diagram illustrating the binding of puerarin to the GABA_{A} (α1β3γ2) receptor, where in the GABA_{A} receptor structure at a resolution of 3.60 A, a drug binding site of puerarin to the GABA_{A} receptor at an atomic level is located between α1 and γ2 subunits of the GABA_{A} receptor, and binding targets for puerarin include H₁₀₂, V₂₀₃-Q₂₀₄-S₂₀₅, T₂₀₇, and Y₂₁₀ of the α1 subunit and D₅₆-M₅₇-Y₅₈, N₆₀, F₇₇, and A₇₉ of the γ2 subunit, which can serve as core drug targets of the GABA_{A} receptor in the preparation of an obesity-treating drug. FIG. 5B shows that the incubation of 0.5 μM GABA in the 293S cell line can cause the GABA_{A} (α1β3γ2) receptor to open a chloride channel, resulting in changes of bioelectric signals (FIG. 5B, left panel). When 0.5 μM GABA was co-incubated with puerarin for 3 min to 5 min, increased chloride ions would enter cells, causing hyperpolarization and making the cells in an inhibition-silent state (FIG. 5B, right panel).

As shown in FIG. 5, cryo-EM analysis results showed that a drug binding site of puerarin to the GABA_{A} receptor at an atomic level was located between the α1/γ2 subunits of the GABA_{A} receptor, and binding targets for puerarin included H₁₀₂, V₂₀₃-Q₂₀₄-S₂₀₅, T₂₀₇, and Y₂₀₉ of the α1 subunit and D₅₆-M₅₇-Y₅₈, N₆₀, F₇₇, and A₇₉ of the γ2 subunit, which may serve as core drug targets of the GABA_{A} receptor in the preparation of an obesity-treating drug.

As shown in FIG. 6, the rAAV-DIO-Gabra1-shRNA (BrainVTA, China) was used to knock down Gabra1 (a gene encoding the α1 subunit of the GABA_{A} receptor) in the DMV in an obese mouse model (obtained through 12 weeks of high-fat feeding), and the viral vector rAAV-DIO-mCherry (BrainVTA, China) without the target gene was used as a control group.

FIG. 6A is a schematic diagram of an experimental process of specific knockdown of Gabra1 to block puerarin: 8-week-old male mice were taken to construct mouse models of alimentary obesity. The mice were fed with a high-fat feed for 8 weeks and then randomly divided into three groups: (1) a control group, which was injected with a control virus into the DMV and intraperitoneally injected with NS 4 weeks later; (2) a puerarin group, which was injected with a control virus into the DMV and intraperitoneally injected with a puerarin solution 4 weeks later; and (3) a Gα1 blocking group, which was injected with a Gabra1shRNA virus into the DMV and then intraperitoneally injected with a puerarin solution 4 weeks later. In the above three animal model groups, before the intraperitoneal injection of puerarin or NS, a body weight was recorded and a feces sample was collected as initial baselines, and this day was set as day 0. From day 1, NS or the puerarin solution was intraperitoneally injected once a day continuously for 7 d, during which a daily body weight change was recorded. 2 h after the NS or the puerarin solution was injected on day 7, the mice were sacrificed, the blood, feces, small intestine, brain, and the like were collected, the physiological indexes such as blood lipid and jejunal TG were quantitatively determined, and the morphological results of the brainstem DMV and small intestine were directly determined through tissue slices.

The c-fos immunofluorescence staining results of brain slices in FIG. 6B showed that the c-fos staining of the puerarin group was less than that of the control group, and neurons in the DMV were inhibited (FIG. 6B, middle panel); the tissue-specific knockdown of Gabra1 (Gα1 blocking group, FIG. 6B, right panel) would intervene in the inhibitory effect of puerarin on neurons in the DMV; and the quantitative signal statistics of c-fos staining for the above three groups in FIG. 6C and Table 8 also showed that the α1 subunit of the GABA_{A} receptor in the DMV played a necessary role in the inhibition of puerarin on neuronal excitability. The control group, the puerarin group, and the Gα1 blocking group were compared in terms of the body weight curve, blood lipid level, and jejunal TG level (FIG. 6D and FIG. 6E (Table 9) and FIG. 6F (Table 10)). Comparison results showed that the DMV tissue-specific knockdown of Gabra1 could block the inhibitory effect of puerarin on the vagus and intervene in the effects of inhibiting the small intestinal lipid absorption, reducing the body weight and the lipid level, indicating that the α1 subunit of the GABA_{A} receptor is a target for puerarin in the treatment of obesity.

TABLE 8
Body weight curve change percentage (%)

| | Day 0 | Day 1 | Day 2 | Day 3 | Day 4 | Day 5 | Day 6 | Day 7 |
|---|---|---|---|---|---|---|---|---|
| Control | 0.00% | 0.00% | 0.39% | 0.51% | 1.02% | 1.27% | 1.27% | 1.65% |
| group-01 | | | | | | | | |
| Control | 0.00% | 0.28% | 0.41% | 0.96% | 1.10% | 1.77% | 2.04% | 2.43% |
| group-02 | | | | | | | | |
| Control | 0.00% | 0.53% | 1.06% | 1.32% | 1.57% | 1.57% | 1.83% | 1.83% |
| group-03 | | | | | | | | |
| Control | 0.00% | 0.14% | 0.69% | 1.23% | 1.50% | 1.64% | 1.77% | 2.17% |
| group-04 | | | | | | | | |
| Control | 0.00% | 0.28% | 0.83% | 0.55% | 1.24% | 1.78% | 1.64% | 2.18% |
| group-05 | | | | | | | | |
| Control | 0.00% | 0.29% | 0.71% | 1.42% | 1.56% | 1.69% | 1.97% | 2.25% |
| group-06 | | | | | | | | |
| Control | 0.00% | 0.00% | 0.27% | 0.14% | 0.14% | 0.27% | 0.27% | 0.41% |
| group-07 | | | | | | | | |
| Control | 0.00% | 0.26% | 0.76% | 1.14% | 1.26% | 1.51% | 1.26% | 1.51% |
| group-08 | | | | | | | | |
| Control | 0.00% | 0.66% | 0.80% | 1.58% | 1.84% | 1.84% | 1.84% | 2.35% |
| group-09 | | | | | | | | |
| Control | 0.00% | 0.26% | 0.13% | 0.65% | 0.78% | 0.39% | 0.52% | 0.13% |
| group-10 | | | | | | | | |
| Mean | 0.00% | 0.27% | 0.60% | 0.95% | 1.20% | 1.37% | 1.44% | 1.69% |
| Puerarin | 0.00% | −0.56% | −2.27% | −2.85% | −2.85% | −4.03% | −4.03% | −4.34% |
| group-01 | | | | | | | | |
| Puerarin | 0.00% | −0.85% | −2.01% | −2.89% | −3.49% | −5.33% | −5.33% | −5.64% |
| group-02 | | | | | | | | |
| Puerarin | 0.00% | −2.20% | −3.33% | −4.79% | −6.90% | −13.76% | −12.05% | −12.73% |
| group-03 | | | | | | | | |
| Puerarin | 0.00% | −1.33% | −2.42% | −4.10% | −5.25% | −6.42% | −8.55% | −8.86% |
| group-04 | | | | | | | | |
| Puerarin | 0.00% | −1.42% | −2.30% | −3.79% | −4.40% | −5.33% | −5.33% | −5.64% |
| group-05 | | | | | | | | |
| Puerarin | 0.00% | −1.08% | −1.08% | −2.45% | −3.30% | −3.01% | −8.67% | −7.43% |
| group-06 | | | | | | | | |
| Puerarin | 0.00% | −0.26% | −2.71% | −3.84% | −4.99% | −5.87% | −6.76% | −6.76% |
| group-07 | | | | | | | | |
| Puerarin | 0.00% | −2.19% | −3.32% | −4.19% | −3.32% | −3.61% | −4.19% | −5.07% |
| group-08 | | | | | | | | |
| Puerarin | 0.00% | −2.54% | −2.82% | −3.70% | −4.30% | −7.69% | −8.33% | −8.98% |
| group-09 | | | | | | | | |
| Puerarin | 0.00% | −1.61% | −1.61% | −3.28% | −4.42% | −4.71% | −5.00% | −5.29% |
| group-10 | | | | | | | | |
| Mean | 0.00% | −1.40% | −2.39% | −3.59% | −4.32% | −5.98% | −6.82% | −7.07% |
| Gα1 | 0.00% | 0.28% | 0.70% | 1.25% | 1.93% | 1.66% | 2.34% | 2.87% |
| blocking | | | | | | | | |
| group-01 | | | | | | | | |
| Gα1 | 0.00% | 0.14% | 0.68% | 0.55% | 0.95% | 0.95% | 1.75% | 1.36% |
| blocking | | | | | | | | |
| group-02 | | | | | | | | |
| Gα1 | 0.00% | 0.00% | 0.80% | 1.33% | 2.36% | 1.85% | 2.36% | 2.87% |
| blocking | | | | | | | | |
| group-03 | | | | | | | | |
| Gα1 | 0.00% | 0.55% | 0.69% | 0.00% | 2.30% | 2.70% | 1.90% | 2.96% |
| blocking | | | | | | | | |
| group-04 | | | | | | | | |
| Gα1 | 0.00% | 0.53% | 1.06% | 1.33% | 1.85% | 1.59% | 1.59% | 1.85% |
| blocking | | | | | | | | |
| group-05 | | | | | | | | |
| Gα1 | 0.00% | 0.41% | 0.68% | 0.95% | 1.21% | 0.68% | 1.21% | 1.21% |
| blocking | | | | | | | | |
| group-06 | | | | | | | | |
| Gα1 | 0.00% | 0.80% | 1.06% | 1.72% | 1.85% | 2.23% | 2.23% | 2.62% |
| blocking | | | | | | | | |
| group-07 | | | | | | | | |
| Gα1 | 0.00% | 0.65% | 0.91% | 1.55% | 1.68% | 1.93% | 1.93% | 2.56% |
| blocking | | | | | | | | |
| group-08 | | | | | | | | |
| Gα1 | 0.00% | 0.14% | 0.82% | 0.69% | 0.96% | 0.96% | 1.23% | 0.96% |
| blocking | | | | | | | | |
| group-09 | | | | | | | | |
| Gα1 | 0.00% | 0.69% | 0.83% | 0.97% | 1.37% | 1.10% | 1.37% | 1.64% |
| blocking | | | | | | | | |
| group-10 | | | | | | | | |
| Mean | 0.00% | 0.42% | 0.82% | 1.03% | 1.65% | 1.56% | 1.79% | 2.09% |

TABLE 9
Blood lipid level (mM)

| Control group | | Puerarin group | | Gα1 blocking group | |
|---|---|---|---|---|---|
| Control group- | 1.0262 | Puerarin | 0.7726 | Gα1 blocking- | 0.9340 |
| 01 | | group-01 | | 01 | |
| Control group- | 0.8533 | Puerarin | 0.6457 | Gα1 blocking- | 0.9801 |
| 02 | | group-02 | | 02 | |
| Control group- | 1.0262 | Puerarin | 0.8187 | Gα1 blocking- | 1.4759 |
| 03 | | group-03 | | 03 | |
| Control group- | 0.8763 | Puerarin | 0.7610 | Gα1 blocking- | 2.1101 |
| 04 | | group-04 | | 04 | |
| Control group- | 0.9801 | Puerarin | 0.7380 | Gα1 blocking- | 1.0608 |
| 05 | | group-05 | | 05 | |
| Control group- | 1.1646 | Puerarin | 0.7034 | Gα1 blocking- | 1.2453 |
| 06 | | group-06 | | 06 | |
| Control group- | 1.3376 | Puerarin | 0.7380 | Gα1 blocking- | 2.1101 |
| 07 | | group-07 | | 07 | |
| Control group- | 1.0493 | Puerarin | 0.8187 | Gα1 blocking- | 1.6373 |
| 08 | | group-08 | | 08 | |
| Control group- | 1.2453 | Puerarin | 0.7841 | Gα1 blocking- | 1.0954 |
| 09 | | group-09 | | 09 | |
| Control group- | 1.1646 | Puerarin | 0.8071 | Gα1 blocking- | 1.1069 |
| 10 | | group-10 | | 10 | |
| Mean | 1.0723 | Mean | 0.7587 | Mean | 1.3756 |

TABLE 10
Jejunal TG level (μmol/mg/protein)

| Control group | | Puerarin group | | Gα1 blocking group | |
|---|---|---|---|---|---|
| Control group- | 0.7671 | Puerarin- | 0.5455 | Gα1 blocking- | 0.6847 |
| 01 | | 01 | | 01 | |
| Control group- | 0.6543 | Puerarin- | 0.6267 | Gα1 blocking- | 0.9986 |
| 02 | | 02 | | 02 | |
| Control group- | 1.1093 | Puerarin- | 0.5654 | Gα1 blocking- | 0.9011 |
| 03 | | 03 | | 03 | |
| Control group- | 0.7019 | Puerarin- | 0.6605 | Gα1 blocking- | 0.7483 |
| 04 | | 04 | | 04 | |
| Control group- | 0.7861 | Puerarin- | 0.3660 | Gα1 blocking- | 0.7285 |
| 05 | | 05 | | 05 | |
| Control group- | 0.7462 | Puerarin- | 0.5925 | Gα1 blocking- | 0.8208 |
| 06 | | 06 | | 06 | |
| Control group- | 1.0606 | Puerarin- | 0.6435 | Gα1 blocking- | 0.7098 |
| 07 | | 07 | | 07 | |
| Control group- | 0.7908 | Puerarin- | 0.6641 | Gα1 blocking- | 1.0251 |
| 08 | | 08 | | 08 | |
| Control group- | 0.6627 | Puerarin- | 0.6873 | Gα1 blocking- | 0.9637 |
| 09 | | 09 | | 09 | |
| Control group- | 0.7473 | Puerarin- | 0.4879 | Gα1 blocking- | 1.1176 |
| 10 | | 10 | | 10 | |
| Mean | 0.8026 | Mean | 0.5839 | Mean | 0.8698 |

Similar to the above experiment, the rAAV-DIO-Gabrg2-shRNA (BrainVTA, China) was used to knock down Gabrg2 (a gene encoding the γ2 subunit of the GABA_{A} receptor) in the DMV in an obese mouse model, and the rAAV-DIO-mCherry (BrainVTA, China) was used as a control group. FIG. 7A is a schematic diagram of an experimental process of specific knockdown of Gabrg2 to block puerarin: 8-week-old male mice were taken to construct mouse models of alimentary obesity. The mice were fed with a high-fat feed for 8 weeks and then randomly divided into three groups: (1) a control group: which was injected with the control virus into the DMV and intraperitoneally injected with NS 4 weeks later; (2) a puerarin group: which was injected with a control virus into the DMV and intraperitoneally injected with the puerarin solution 4 weeks later; and (3) a Gγ2 blocking group: which was injected with a Gabrg2 shRNA virus into the DMV and then intraperitoneally injected with a puerarin solution 4 weeks later. In the above three animal model groups, before the intraperitoneal injection of puerarin or NS, a body weight was recorded and a feces sample was collected as initial baselines, and this day was set as day 0. From day 1, NS or the puerarin solution was intraperitoneally injected once a day continuously for 7 d, during which a daily body weight change was recorded. 2 h after the NS or the puerarin solution injection on day 7, the mice were sacrificed, the blood, feces, small intestine, brain, and the like were collected, the physiological indexes such as blood lipid and jejunal TG were quantitatively determined, and the morphological results of the brainstem DMV and small intestine were directly determined through tissue slices.

The c-fos staining results of DMV in the control group, puerarin group, and Gγ2 blocking group (FIG. 7B) were compared and subjected to quantitative statistical analysis (FIG. 7C, Table 11). According to the quantitative statistics in FIG. 7C, a c-fos staining signal of neurons in the DMV was inhibited by puerarin; and according to FIG. 7B, the tissue-specific knockdown of Gabrg2 could intervene in the inhibitory effect of puerarin on neurons in the DMV. A body weight curve after the intraperitoneal injection of puerarin was recorded (FIG. 7D) and the blood lipid level (FIG. 7E, Table 12) and jejunal TG level (FIG. 7F, Table 13) were measured. It was showed that the DMV tissue-specific knockdown of Gabrg2 could block the inhibitory effect of puerarin on the vagus and intervene in the effects of inhibiting the small intestinal lipid absorption, reducing the body weight, and reducing the lipid level, indicating that the γ2 subunit of the GABA_{A} receptor is a target for puerarin in the treatment of obesity.

TABLE 11
Body weight curve change percentage (%)

| | Day 0 | Day 1 | Day 2 | Day 3 | Day 4 | Day 5 | Day 6 | Day 7 |
|---|---|---|---|---|---|---|---|---|
| Control | 0.00% | 0.00% | 1.34% | 2.13% | 1.87% | 1.34% | 1.61% | 2.39% |
| group-01 | | | | | | | | |
| Control | 0.00% | −0.57% | −0.57% | 0.56% | −0.85% | −0.28% | −0.28% | 0.28% |
| group-02 | | | | | | | | |
| Control | 0.00% | 0.00% | 0.26% | 1.29% | 2.05% | 1.29% | 1.03% | 1.79% |
| group-03 | | | | | | | | |
| Control | 0.00% | 0.28% | 0.00% | 0.57% | 0.57% | 0.85% | 1.40% | 0.85% |
| group-04 | | | | | | | | |
| Control | 0.00% | 0.00% | 0.27% | 0.79% | 1.31% | 1.31% | 0.79% | 1.31% |
| group-05 | | | | | | | | |
| Mean | 0.00% | −0.06% | 0.26% | 1.07% | 0.99% | 0.90% | 0.91% | 1.33% |
| Puerarin | 0.00% | −1.10% | −0.82% | −2.50% | −3.07% | −4.83% | −5.13% | −6.34% |
| group-01 | | | | | | | | |
| Puerarin | 0.00% | −1.06% | −1.60% | −0.53% | −2.14% | −2.97% | −4.10% | −4.96% |
| group-02 | | | | | | | | |
| Puerarin | 0.00% | −0.81% | −0.81% | −2.20% | −2.76% | −1.64% | −3.05% | −3.33% |
| group-03 | | | | | | | | |
| Puerarin | 0.00% | −0.79% | −1.06% | −1.33% | −1.87% | −2.97% | −5.54% | −5.54% |
| group-04 | | | | | | | | |
| Puerarin | 0.00% | 0.82% | −1.11% | −1.68% | −0.83% | −5.20% | −3.41% | −3.41% |
| group-05 | | | | | | | | |
| Mean | 0.00% | −0.59% | −1.08% | −1.65% | −2.14% | −3.52% | −4.24% | −4.72% |
| Gγ2 | 0.00% | 0.82% | 1.37% | 0.55% | 0.28% | 0.55% | 0.55% | 0.82% |
| blocking | 0.00% | 0.55% | −0.28% | −1.13% | −1.41% | −0.56% | −0.56% | 0.00% |
| group-01 | | | | | | | | |
| Gγ2 | | | | | | | | |
| blocking | | | | | | | | |
| group-02 | | | | | | | | |
| Gγ2 | 0.00% | −0.82% | 0.27% | 0.54% | 0.80% | 2.62% | 2.62% | 2.37% |
| blocking | 0.00% | −0.55% | 0.54% | 1.08% | 1.60% | 3.41% | 3.41% | 3.16% |
| group-03 | | | | | | | | |
| Gγ2 | | | | | | | | |
| blocking | 0.00% | 0.79% | −0.81% | 1.57% | 1.06% | 0.79% | 1.83% | 2.09% |
| group-04 | | | | | | | | |
| Gγ2 | | | | | | | | |
| blocking | | | | | | | | |
| group-05 | | | | | | | | |
| Mean | 0.00% | 0.16% | 0.22% | 0.52% | 0.47% | 1.36% | 1.57% | 1.69% |

TABLE 12
Blood lipid level (mM)

| Control group | | Puerarin group | | Gγ2 blocking group | |
|---|---|---|---|---|---|
| Control group- | 1.2622 | Puerarin- | 0.8673 | Gγ2 blocking- | 0.9340 |
| 01 | | 01 | | 01 | |
| Control group- | 1.4659 | Puerarin- | 0.7337 | Gγ2 blocking- | 0.9801 |
| 02 | | 02 | | 02 | |
| Control group- | 1.3376 | Puerarin- | 0.7380 | Gγ2 blocking- | 1.4759 |
| 03 | | 03 | | 03 | |
| Control group- | 1.3453 | Puerarin- | 0.6408 | Gγ2 blocking- | 2.1101 |
| 04 | | 04 | | 04 | |
| Control group- | 1.1646 | Puerarin- | 0.7143 | Gγ2 blocking- | 1.0608 |
| 05 | | 05 | | 05 | |
| Mean | 1.3151 | Mean | 0.7388 | Mean | 1.3122 |

TABLE 13
Jejunal TG level (μmol/mg/protein)

| Control group | | Puerarin group | | Gγ2 blocking group | |
|---|---|---|---|---|---|
| Control group- | 1.0292 | Puerarin- | 0.8466 | Gγ2 blocking- | 1.6522 |
| 01 | | 01 | | 01 | |
| Control group- | 1.1863 | Puerarin- | 0.9139 | Gγ2 blocking- | 1.1566 |
| 02 | | 02 | | 02 | |
| Control group- | 1.3334 | Puerarin- | 0.7516 | Gγ2 blocking- | 1.0093 |
| 03 | | 03 | | 03 | |
| Control group- | 1.3316 | Puerarin- | 0.9191 | Gγ2 blocking- | 1.1912 |
| 04 | | 04 | | 04 | |
| Control group- | 1.3674 | Puerarin- | 0.8860 | Gγ2 blocking- | 1.5075 |
| 05 | | 05 | | 05 | |
| Mean | 1.2496 | Mean | 0.8634 | Mean | 1.3034 |

In summary, the α1 and γ2 subunits of the GABA_{A} receptor in the DMV are drug targets for puerarin to reduce body weight and lipid; and the GABA_{A} receptor in the DMV can be widely used as a drug target for reducing blood lipid, treating obesity, and improving metabolism. The present disclosure claims a use of the GABA_{A} receptor in the DMV as a drug target for treating obesity.

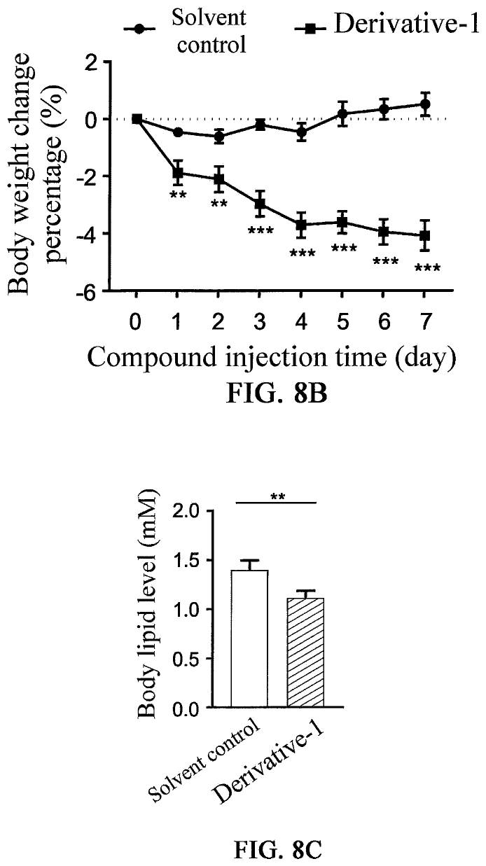

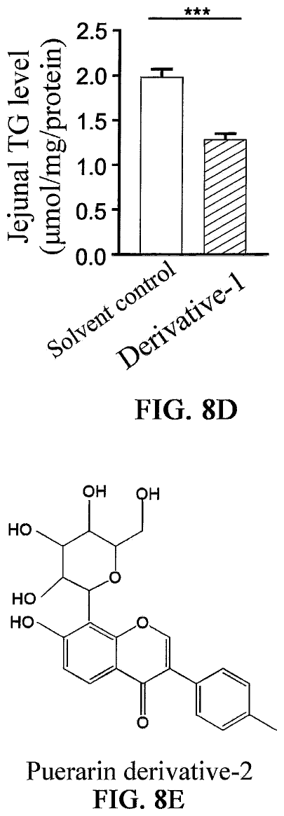

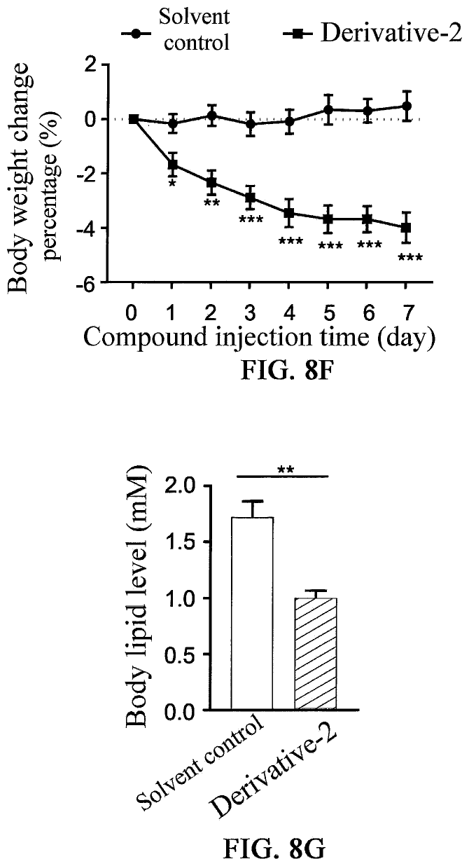

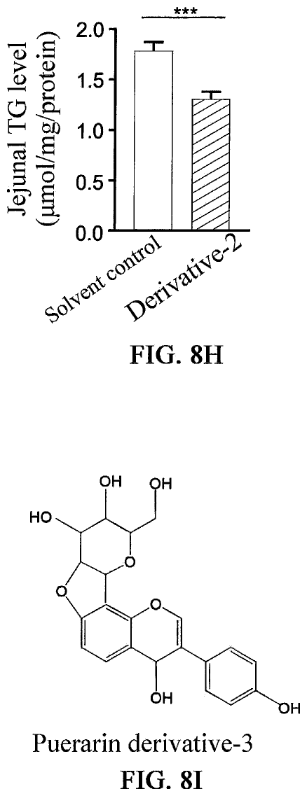

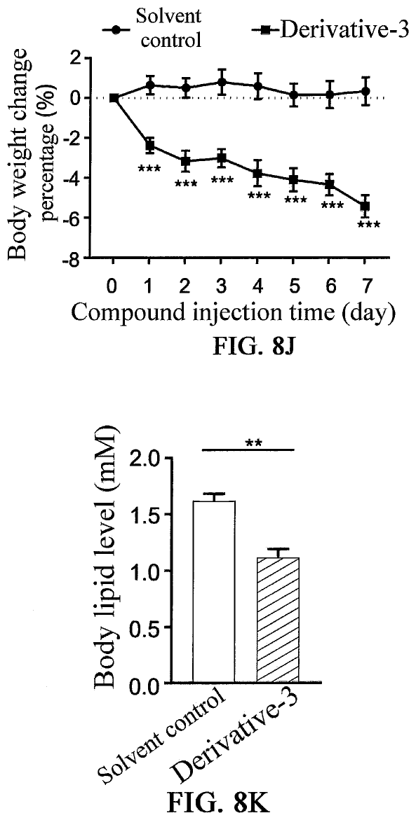

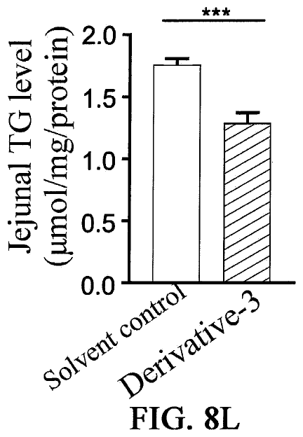

In the present disclosure, puerarin derivatives were prepared by a chemical synthesis method (Lou Hongxiang, Sun Bin, Cui Changyi. Puerarin Derivative, Preparation Method thereof, and Use thereof in Prevention and Treatment of Cardiovascular Disease (CVD) or Diabetes and Complications thereof [P]: CN201710464386.5, 20200228; Wang Lin, Zhang Shouguo, Peng Tao, Lv Qiujun. Synthesis and Preliminary activity investigation of Puerarin and derivative thereof [A]. in: 2005 National Pharmacochemical Conference [C]. 2005. 187-187; Zhang Bin. Synthesis Investigation of Puerarin Derivative and Pyrazole Compound Converted therefrom [D]. Shaanxi University of Science and Technology, 2016), and the effects of each derivative in weight-reducing, lipid-lowering, and obesity-treating were experimentally demonstrated. As shown in FIG. 8A to FIG. 8D and Tables 14 to 16, Intraperitoneally injection of the puerarin derivative-1 could significantly reduce the body weight of the obese mouse (FIG. 8B), inhibit the TG absorption in small intestine (FIG. 8D), and effectively reduce blood lipid level (FIG. 8C). The puerarin derivative-2 and the puerarin derivative-3 both exhibit the effects of inhibiting the small intestinal lipid absorption, and reducing blood lipid and body weight (FIG. 8 E to FIG. 8L, and Tables 17 to 22).

TABLE 14
Body weight change percentage for the puerarin derivative-1 group (%)

| | Day 0 | Day 1 | Day 2 | Day 3 | Day 4 | Day 5 | Day 6 | Day 7 |
|---|---|---|---|---|---|---|---|---|
| Control group-01 | 0.00% | −0.32% | −0.32% | 0.63% | 0.00% | 2.47% | 1.25% | −0.32% |
| Control group-02 | 0.00% | −0.59% | −0.29% | −0.88% | −0.88% | 1.44% | 0.00% | 0.58% |
| Control group-03 | 0.00% | 0.00% | 0.23% | 0.23% | 0.23% | 0.90% | 1.56% | 2.85% |
| Control group-04 | 0.00% | −0.84% | −0.56% | −0.84% | 0.00% | 1.36% | 1.09% | 1.90% |
| Control group-05 | 0.00% | −0.27% | −1.34% | −0.27% | −0.27% | −0.80% | −0.80% | −0.53% |
| Control group-06 | 0.00% | 0.24% | −0.24% | −0.24% | −0.95% | −1.20% | −0.95% | −1.44% |
| Control group-07 | 0.00% | −0.25% | −0.50% | 0.25% | 1.23% | 0.50% | 2.19% | 1.23% |
| Control group-08 | 0.00% | −1.38% | 0.23% | 0.23% | −0.23% | −0.68% | 0.23% | −0.23% |
| Control group-09 | 0.00% | −0.52% | −1.04% | −0.78% | −2.10% | −1.30% | −0.52% | 0.76% |
| Control group-10 | 0.00% | −0.63% | −2.25% | −0.32% | −1.60% | −0.95% | −0.63% | 0.31% |
| Mean | 0.00% | −0.45% | −0.61% | −0.20% | −0.46% | 0.17% | 0.34% | 0.51% |
| Derivative 1-01 | 0.00% | −0.25% | −0.74% | −1.24% | −0.99% | −1.74% | −4.08% | −3.81% |
| Derivative 1-02 | 0.00% | −2.34% | −1.86% | −3.06% | −3.79% | −3.55% | −3.06% | −2.10% |
| Derivative 1-03 | 0.00% | −2.27% | −2.56% | −2.56% | −3.73% | −4.33% | −2.56% | −5.25% |
| Derivative 1-04 | 0.00% | −3.30% | −3.79% | −4.04% | −6.05% | −6.05% | −6.31% | −6.56% |
| Derivative 1-05 | 0.00% | −2.93% | −4.32% | −4.32% | −4.88% | −3.48% | −5.17% | −6.62% |
| Derivative 1-06 | 0.00% | 0.00% | −0.26% | −1.55% | −3.15% | −2.08% | −2.35% | −2.35% |
| Derivative 1-07 | 0.00% | −2.45% | −2.20% | −2.45% | −3.46% | −2.95% | −3.21% | −2.20% |
| Derivative 1-08 | 0.00% | 0.28% | 0.00% | −1.14% | −2.30% | −3.79% | −2.60% | −3.79% |
| Derivative 1-09 | 0.00% | −2.89% | −2.62% | −4.53% | −4.26% | −3.70% | −5.09% | −4.26% |
| Derivative 1-10 | 0.00% | −2.63% | −2.63% | −4.77% | −4.46% | −4.46% | −5.08% | −3.84% |
| Mean | 0.00% | −1.88% | −2.10% | −2.96% | −3.71% | −3.61% | −3.95% | −4.08% |

TABLE 15
Blood lipid level for the puerarin derivative-1 group (mM)

| | |
|---|---|
| Control group | Derivative-1 |

| | | | |
|---|---|---|---|
| Control group-01 | 1.2385 | Derivative 1-01 | 1.0385 |
| Control group-02 | 1.8077 | Derivative 1-02 | 0.7869 |
| Control group-03 | 1.7462 | Derivative 1-03 | 0.9462 |
| Control group-04 | 1.8923 | Derivative 1-04 | 1.0538 |
| Control group-05 | 1.4385 | Derivative 1-05 | 1.1154 |
| Control group-06 | 1.2154 | Derivative 1-06 | 1.4538 |
| Control group-07 | 1.2000 | Derivative 1-07 | 0.9077 |
| Control group-08 | 1.2462 | Derivative 1-08 | 1.0615 |
| Control group-09 | 1.1308 | Derivative 1-09 | 0.8154 |
| Control group-10 | 1.0923 | Derivative 1-10 | 1.3000 |
| Mean | 1.4008 | Mean | 1.0479 |

TABLE 16
Jejunal TG level for the puerarin derivative-1
group (μmol/mg/protein)

| | |
|---|---|
| Control group | Derivative-1 |

| | | | |
|---|---|---|---|
| Control group-01 | 2.0371 | Derivative 1-01 | 1.3121 |
| Control group-02 | 1.8118 | Derivative 1-02 | 1.0924 |
| Control group-03 | 1.7667 | Derivative 1-03 | 1.2506 |
| Control group-04 | 1.7814 | Derivative 1-04 | 0.9988 |
| Control group-05 | 2.3289 | Derivative 1-05 | 1.5325 |
| Control group-06 | 2.1159 | Derivative 1-06 | 1.5551 |
| Control group-07 | 2.2167 | Derivative 1-07 | 1.3572 |
| Control group-08 | 2.1148 | Derivative 1-08 | 1.5063 |
| Control group-09 | 1.4556 | Derivative 1-09 | 1.1252 |
| Control group-10 | 2.2141 | Derivative 1-10 | 1.1433 |
| Mean | 1.9843 | Mean | 1.2874 |

TABLE 17
Body weight change curve for the puerarin derivative-2 group (%)

| | Day 0 | Day 1 | Day 2 | Day 3 | Day 4 | Day 5 | Day 6 | Day 7 |
|---|---|---|---|---|---|---|---|---|
| Control | 0.00% | 0.68% | 0.53% | 0.89% | 1.53% | 2.61% | 2.18% | 2.54% |
| group-01 | | | | | | | | |
| Control | 0.00% | 0.34% | −1.03% | −1.52% | −0.91% | −1.03% | −0.02% | −1.03% |
| group-02 | | | | | | | | |
| Control | 0.00% | −0.78% | −0.39% | −0.78% | −0.78% | −0.78% | −0.78% | −0.78% |
| group-03 | | | | | | | | |
| Control | 0.00% | −2.54% | −0.31% | −1.90% | −2.22% | −2.54% | −2.54% | −2.54% |
| group-04 | | | | | | | | |
| Control | 0.00% | 0.31% | 1.52% | 1.52% | 1.81% | 0.61% | 0.92% | 1.52% |
| group-05 | | | | | | | | |
| Control | 0.00% | −0.65% | −1.63% | −1.63% | −1.30% | −1.30% | −0.32% | −0.32% |
| group-06 | | | | | | | | |
| Control | 0.00% | 1.42% | 2.45% | 1.77% | 1.77% | 0.71% | 0.71% | 1.42% |
| group-07 | | | | | | | | |
| Control | 0.00% | 0.32% | 0.32% | 0.65% | 0.00% | 2.54% | 1.29% | 0.32% |
| group-08 | | | | | | | | |
| Control | 0.00% | −0.68% | −0.34% | −1.03% | −1.03% | 1.67% | 0.00% | 0.67% |
| group-09 | | | | | | | | |
| Control | 0.00% | 0.00% | 0.25% | 0.25% | 0.25% | 0.98% | 1.70% | 3.10% |
| group-10 | | | | | | | | |
| Mean | 0.00% | −0.16% | 0.14% | −0.18% | −0.09% | 0.35% | 0.31% | 0.49% |
| Derivative | 0.00% | −2.39% | −2.74% | −2.74% | −2.74% | −2.74% | −2.74% | −3.45% |
| 2-01 | | | | | | | | |
| Derivative | 0.00% | −0.56% | −2.26% | −3.43% | −3.73% | −4.03% | −4.03% | −2.26% |
| 2-02 | | | | | | | | |
| Derivative | 0.00% | −1.13% | −1.99% | −3.16% | −3.16% | −3.76% | −3.16% | −2.57% |
| 2-03 | | | | | | | | |
| Derivative | 0.00% | −2.22% | −4.21% | −4.55% | −3.87% | −4.21% | −3.54% | −3.87% |
| 2-04 | | | | | | | | |
| Derivative | 0.00% | 0.90% | 0.60% | 0.00% | −0.91% | −0.61% | −0.91% | −2.15% |
| 2-05 | | | | | | | | |
| Derivative | 0.00% | −2.19% | −2.75% | −2.75% | −3.89% | −3.89% | −4.47% | −5.66% |
| 2-06 | | | | | | | | |
| Derivative | 0.00% | −0.28% | −0.84% | −1.41% | −1.13% | −1.99% | −4.67% | −4.37% |
| 2-07 | | | | | | | | |
| Derivative | 0.00% | −2.70% | −2.15% | −3.55% | −4.40% | −4.11% | −3.55% | −2.43% |
| 2-08 | | | | | | | | |
| Derivative | 0.00% | −2.55% | −2.88% | −2.88% | −4.21% | −4.89% | −2.88% | −5.93% |
| 2-09 | | | | | | | | |
| Derivative | 0.00% | −3.57% | −4.10% | −4.37% | −6.57% | −6.57% | −6.85% | −7.13% |
| 2-10 | | | | | | | | |
| Mean | 0.00% | −1.67% | −2.33% | −2.88% | −3.46% | −3.68% | −3.68% | −3.98% |

TABLE 18
Blood lipid level for the puerarin derivative-2 group (mM)

| | |
|---|---|
| Control group | Derivative-2 |

| | | | |
|---|---|---|---|
| Control group-01 | 1.8345 | Derivative 2-01 | 1.3428 |
| Control group-02 | 1.2766 | Derivative 2-02 | 0.9551 |
| Control group-03 | 1.4373 | Derivative 2-03 | 1.1631 |
| Control group-04 | 1.9196 | Derivative 2-04 | 1.1347 |
| Control group-05 | 0.9551 | Derivative 2-05 | 1.0307 |
| Control group-06 | 1.5224 | Derivative 2-06 | 1.0874 |
| Control group-07 | 2.2127 | Derivative 2-07 | 1.0496 |
| Control group-08 | 2.4397 | Derivative 2-08 | 0.6903 |
| Control group-09 | 2.0614 | Derivative 2-09 | 0.8792 |
| Control group-10 | 1.5352 | Derivative 2-10 | 0.6729 |
| Mean | 1.7194 | Mean | 1.0006 |

TABLE 19
Jejunal TG level for the puerarin derivative-2
group (μmol/mg/protein)

| | |
|---|---|
| Control group | Derivative-2 |

| | | | |
|---|---|---|---|
| Control group-01 | 1.3198 | Derivative 2-01 | 1.3121 |
| Control group-02 | 1.7874 | Derivative 2-02 | 1.0924 |
| Control group-03 | 1.5283 | Derivative 2-03 | 1.2506 |
| Control group-04 | 1.7106 | Derivative 2-04 | 0.9988 |
| Control group-05 | 2.0371 | Derivative 2-05 | 1.7043 |
| Control group-06 | 1.8118 | Derivative 2-06 | 1.5325 |
| Control group-07 | 1.7667 | Derivative 2-07 | 1.5551 |
| Control group-08 | 1.8123 | Derivative 2-08 | 1.3572 |
| Control group-09 | 1.7814 | Derivative 2-09 | 1.1252 |
| Control group-10 | 2.3289 | Derivative 2-10 | 1.1433 |
| Mean | 1.7884 | Mean | 1.3072 |

TABLE 20
Body weight change curve for the puerarin derivative-3 group (%)

| | Day 0 | Day 1 | Day 2 | Day 3 | Day 4 | Day 5 | Day 6 | Day 7 |
|---|---|---|---|---|---|---|---|---|
| Control | 0.00% | −0.83% | −1.68% | −2.25% | −2.83% | −2.54% | −1.68% | −2.25% |
| group-01 | | | | | | | | |
| Control | 0.00% | −0.89% | −1.20% | −1.50% | −1.50% | −1.50% | −3.36% | −3.04% |
| group-02 | | | | | | | | |
| Control | 0.00% | 2.69% | 2.42% | 2.95% | 2.16% | −0.83% | 2.16% | 2.16% |
| group-03 | | | | | | | | |
| Control | 0.00% | 0.28% | 1.09% | 1.63% | 0.82% | 1.09% | 0.00% | 1.09% |
| group-04 | | | | | | | | |
| Control | 0.00% | 0.83% | 0.28% | 0.83% | 0.83% | 0.83% | 0.83% | 0.83% |
| group-05 | | | | | | | | |
| Control | 0.00% | 2.39% | 0.27% | 1.87% | 2.13% | 2.39% | 2.39% | 2.39% |
| group-06 | | | | | | | | |
| Control | 0.00% | 0.27% | 1.32% | 1.32% | 1.84% | 0.53% | 0.80% | 1.32% |
| group-07 | | | | | | | | |
| Control | 0.00% | 0.54% | 1.61% | 1.61% | 1.34% | 1.34% | 0.27% | 0.27% |
| group-08 | | | | | | | | |
| Mean | 0.00% | 0.66% | 0.51% | 0.81% | 0.60% | 0.16% | 0.18% | 0.35% |
| Derivative | 0.00% | −3.96% | −5.57% | −1.49% | −2.09% | −3.02% | −3.33% | −5.24% |
| 3-01 | | | | | | | | |
| Derivative | 0.00% | −2.50% | −1.65% | −2.50% | −3.65% | −3.65% | −5.13% | −6.65% |
| 3-02 | | | | | | | | |
| Derivative | 0.00% | −1.71% | −3.77% | −4.68% | −6.24% | −6.24% | −6.24% | −7.51% |
| 3-03 | | | | | | | | |
| Derivative | 0.00% | −2.35% | −2.65% | −2.65% | −2.65% | −2.65% | −2.65% | −3.26% |
| 3-04 | | | | | | | | |
| Derivative | 0.00% | −2.28% | −4.06% | −4.36% | −3.76% | −4.06% | −3.46% | −3.76% |
| 3-05 | | | | | | | | |
| Derivative | 0.00% | −0.26% | −0.80% | −1.34% | −1.07% | −1.88% | −4.41% | −4.13% |
| 3-06 | | | | | | | | |
| Derivative | 0.00% | −2.59% | −2.88% | −2.88% | −4.39% | −5.00% | −2.88% | −5.93% |
| 3-07 | | | | | | | | |
| Derivative | 0.00% | −3.32% | −3.90% | −4.19% | −6.27% | −6.27% | −6.57% | −6.88% |
| 3-08 | | | | | | | | |
| Mean | 0.00% | −2.37% | −3.16% | −3.01% | −3.76% | −4.10% | −4.33% | −5.42% |

TABLE 21
Blood lipid level for the puerarin derivative-3 group (mM)

| | |
|---|---|
| Control group | Derivative-3 |

| | | | |
|---|---|---|---|
| Control group-01 | 1.61 | Derivative 3-01 | 1.06 |
| Control group-02 | 1.92 | Derivative 3-02 | 1.23 |
| Control group-03 | 1.87 | Derivative 3-03 | 1.07 |
| Control group-04 | 1.55 | Derivative 3-04 | 1.29 |
| Control group-05 | 1.62 | Derivative 3-05 | 1.18 |
| Control group-06 | 1.48 | Derivative 3-06 | 1.62 |
| Control group-07 | 1.47 | Derivative 3-07 | 1.06 |
| Control group-08 | 1.42 | Derivative 3-08 | 1.69 |
| Mean | 1.6179 | Mean | 1.2750 |

TABLE 22
Jejunal TG level for the puerarin derivative-3
group (μmol/mg/protein)

| | |
|---|---|
| Control group | Derivative-3 |

| | | | |
|---|---|---|---|
| Control group-01 | 1.92 | Derivative 3-01 | 1.48 |
| Control group-02 | 1.73 | Derivative 3-02 | 1.53 |
| Control group-03 | 1.58 | Derivative 3-03 | 1.06 |
| Control group-04 | 1.61 | Derivative 3-04 | 0.85 |
| Control group-05 | 1.83 | Derivative 3-05 | 1.29 |
| Control group-06 | 1.61 | Derivative 3-06 | 1.48 |
| Control group-07 | 1.94 | Derivative 3-07 | 1.36 |
| Control group-08 | 1.85 | Derivative 3-08 | 1.27 |
| Mean | 1.7588 | Mean | 1.2900 |

In conclusion, the GABA_{A} receptor may inhibit the small intestinal lipid absorption through the pathway of “DMV-GABA_{A} receptor-vagus-small intestinal lipid absorption”, thereby achieving the goals of reducing blood lipid and body weight, and treating obesity. As a GABA_{A} receptor agonist, the puerarin (and a derivative thereof) involved in the present disclosure may promote the opening of a chloride channel and inhibit the neuronal activity of the DMV, thereby achieving the goals of promoting the body's lipid excretion and lowering lipid level.

The above GABA_{A} receptor-regulated brain-gut axis mechanism may be used as a target for preparing an obesity-treating drug.

Although the present disclosure has been described in detail through the above examples, the examples are only a part rather than all of the embodiments of the present disclosure. All other embodiments obtained by a person based on these examples without creative efforts shall fall within a protection scope of the present disclosure.

What is claimed is:

1. A method for treating an obesity-related disease, comprising administering a therapeutically effective dose of a drug that targets a GABA_{A} receptor to a subject in need thereof, wherein the obesity-related disease is selected from the group consisting of (1) hyperlipidemia; (2) obesity; (3) metabolic syndrome; and (4) excessive absorption of small intestinal lipid.

2. The method according to claim 1, wherein the drug comprises a substance for increasing an expression level of the GABA_{A} receptor.

3. The method according to claim 1, wherein the drug comprises a substance for improving sensitivity of the GABA_{A} receptor, improving a chloride channel opening frequency, increasing a chloride influx, or causing nerve cell hyperpolarization.

4. The method according to claim 1, wherein the drug comprises a GABA_{A} receptor agonist and/or allosteric modulator.

5. The method according to claim 4, wherein the GABA_{A} receptor agonist comprises puerarin and/or a derivative thereof, or a benzodiazepine, wherein the benzodiazepine comprises one or more selected from the group consisting of flunitrazepam, diazepam, triazolam, and flumazenil.

6. The method according to claim 1, wherein the drug comprises a substance for inhibiting nerve excitability of a dorsal motor nucleus of the vagus (DMV).

7. The method according to claim 6, wherein the substance for inhibiting nerve excitability of the DMV comprises a substance for increasing an expression level of the GABA_{A} receptor or a substance for improving sensitivity of the GABA_{A} receptor or a chloride channel opening frequency, increasing a chloride influx, or causing nerve cell hyperpolarization.

8. The method according to claim 1, wherein the drug comprises a substance that targets a gene Gabra1 and/or a gene Gabrg2.

9. A method for treating an obesity-related disease, comprising administering a therapeutically effective dose of puerarin or derivatives thereof to a subject in need thereof, wherein the obesity-related disease is selected from the group consisting of (1) hyperlipidemia; (2) obesity; (3) metabolic syndrome; and (4) excessive absorption of small intestinal lipid.

10. A method for screening a drug for treating an obesity-related disease, comprising selecting a drug that targets a GABA_{A} receptor, wherein the obesity-related disease is selected from the group consisting of (1) hyperlipidemia; (2) obesity; (3) metabolic syndrome; and (4) excessive absorption of small intestinal lipid.

11. The method according to claim 10, wherein the drug comprises a substance for increasing an expression level of the GABA_{A} receptor.

12. The method according to claim 10, wherein the drug comprises a substance for improving sensitivity of the GABA_{A} receptor, improving a chloride channel opening frequency, increasing a chloride influx, or causing nerve cell hyperpolarization.

13. The method according to claim 10, wherein the drug comprises a GABA_{A} receptor agonist and/or allosteric modulator.

14. The method according to claim 13, wherein the GABA_{A} receptor agonist comprises puerarin and/or a derivative thereof, or a benzodiazepine; and the benzodiazepine comprises one or more selected from the group consisting of flunitrazepam, diazepam, triazolam, and flumazenil.

15. The method according to claim 10, wherein the drug comprises a substance for inhibiting nerve excitability of a DMV.

16. The method according to claim 15, wherein the substance for inhibiting nerve excitability of the DMV comprises a substance for increasing an expression level of a GABA_{A} receptor or a substance for improving sensitivity of the GABA_{A} receptor or a chloride channel opening frequency, increasing a chloride influx, or causing nerve cell hyperpolarization.

17. The method according to claim 10, wherein the drug comprises a substance that targets a gene Gabra1 and/or a gene Gabrg2.
